(19) United States
(12) Patent Application Publication
(10) Pub. No.: US 2022/0326257 A1
(43) Pub. Date: Oct. 13, 2022

(54) DETECTION OF AUTOANTIBODIES AGAINST DEIMINATED PROTEIN EPITOPES ASSOCIATED WITH BRAIN OXYGEN DEPRIVATION

(71) Applicant: THE HENRY M. JACKSON FOUNDATION FOR THE ADVANCEMENT OF MILITARY MEDICINE, INC., Bethesda, MD (US)

(72) Inventor: Gregory P. MUELLER, Silver Spring, MD (US)

(73) Assignee: THE HENRY M. JACKSON FOUNDATION FOR THE ADVANCEMENT OF MILITARY MEDICINE, INC., Bethesda, MD (US)

(21) Appl. No.: 17/051,857

(22) PCT Filed: Apr. 30, 2019
(86) PCT No.: PCT/US2019/029856

§ 371 Date: Oct. 30, 2020

Related U.S. Application Data
(60) Provisional application No. 62/665,919, filed on May 2, 2018.

(51) Int. Cl. G01N 33/68; G01N 33/533

(52) U.S. Cl. CPC G01N 33/6896; G01N 33/533; G01N 2800/2871; G01N 2800/52

(57) ABSTRACT

Described herein are deiminated proteins that are deiminated in response to oxygen-deprivation brain injury (ODBI) and/or oxygen-deprivation causing injury (ODCI). Also described are related methods and devices for detecting, diagnosing, and monitoring ODBI or ODCI, and methods for treating ODBI or ODCI.

### CROSS-REFERENCE TO RELATED APPLICATIONS

This application is the U.S. National Stage of International Application No. PCT/US2019/029856, filed on Apr. 30, 2019, and claims priority to U.S. provisional application 62/665,919, filed May 2, 2018, the entire contents of which are incorporated herein by reference in their entirety.

### STATEMENT OF GOVERNMENT SUPPORT

This invention was made with government support under Intramural Grant 92-447 and USUHS Award APG-70-4247, awarded by Uniformed Services University, DMRDP grant W81XWH-13-C-0196, and DARPA PREVENT grant. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 5, 2021, is named 103783-0271_SL.txt and is 54,617 bytes in size.

### FIELD

Described herein are deiminated proteins and peptides and related devices and methods, useful in detecting and diagnosing an oxygen-deprivation brain injury (ODBI) or oxygen-deprivation causing injury (ODCI).

### BACKGROUND

Oxygen-deprivation brain injury (ODBI) and oxygen-deprivation causing injury (ODCI) may cause long-term disabilities, developmental delays, and death. ODBI includes anoxic brain injury (when the brain is totally deprived of oxygen) such as may occur with sudden cardiac arrest, choking, strangulation, and other sudden injuries, and hypoxic brain injury (when the brain receives an insufficient amount of oxygen).

Traumatic brain injury (TBI) is an example of ODCI and a major cause of long-term disability. Acute TBI prompts a constellation of dysfunctional processes, collectively known as “secondary injury” mechanisms. A hallmark secondary injury in TBI is a prolonged imbalance in calcium homeostasis, resulting in a dramatic influx of calcium into brain cells. This influx elicits the generation of damaging reactive oxygen species. Protein deimination is a pathological post-translational modification that can result from intracellular calcium overload, and has been proposed to play a role in neurodegenerative disorders, including Alzheimer's disease, and multiple sclerosis. Deimination can contribute to ongoing dysfunction, either through direct loss of protein function or via immune-based mechanisms where proteins specifically modified by deimination become targeted by the adaptive immune system.

Central features in traumatic brain injury (TBI) include oxidative stress [1-4], breakdown of the blood brain barrier [5, 6], and a protracted period of Ca²⁺ excitotoxicity [7, 8]. These early consequences of brain injury set the stage for the progressive development of long-term pathologies including impaired learning and memory, as well as emotional and mood imbalances [9-13]. These long-term consequences of TBI can be complex, and may increase in severity over months and years, even though the injury may have been classified as clinically mild and there is no evidence of physical injury using the most sensitive of imaging techniques [14, 15]. At present, there is a gap in our knowledge linking the acute events of mild TBI to chronic pathology. Importantly, repeated mild TBI has now been identified as the most significant environmental factor for developing chronic neuropsychiatric symptoms [16-18].

Thus, there remains a need to identify effective biomarkers of ODBI and ODCI, including TBI, and a need for methods and devices for detecting and diagnosing ODBI and ODCI, including TBI.

### SUMMARY

Disclosed herein are methods of analyzing a biological sample obtained from a subject, comprising detecting antibodies present in the biological sample, wherein the antibodies are specific to a deiminated variant of one or more proteins listed in Table 1 or a fragment thereof. In some embodiments, the antibodies are specific to a deiminated variant of one or more proteins listed in Table 1 or a fragment thereof, wherein the protein or fragment is deiminated at a site(s) listed in Table 1. In some embodiments, the method further comprises detecting antibodies specific to a deiminated variant of one or more proteins listed in Tables 2 and/or Table 3 or a fragment thereof.

Further disclosed herein are methods of analyzing a biological sample obtained from a subject, comprising detecting antibodies present in the biological sample, wherein the antibodies are specific to a deiminated variant of one or more proteins listed in Table 2 or a fragment thereof, wherein the protein or fragment is deiminated at a site(s) listed in Table 2. In some embodiments, the method further comprises detecting antibodies specific to a deiminated variant of one or more proteins listed in Table 3 or a fragment thereof.

In some embodiments, the method comprises detecting a plurality of different antibodies, each specific to a different deiminated variant.

In some embodiments, the method comprises detecting a plurality of different antibodies, each specific to a different deiminated variant, wherein the deiminated variants are deiminated at a listed site(s). In some embodiments, the antibodies are immunoglobulin gamma (IgG) antibodies. In some embodiments, the antibodies are immunoglobulin mu (IgM) antibodies.

In some embodiments, detecting antibodies comprises contacting the biological sample with a deiminated peptide that comprises at least a deiminated portion of the deiminated variant. In some embodiments, the deiminated peptide is deiminated at a listed site(s). In some embodiments, the deiminated peptide comprise from 3 to 25 amino acid residues. In some embodiments, the deiminated peptide is attached to a solid support. In some embodiments, detecting antibodies comprises contacting any antibodies bound to the deiminated peptide with secondary antibodies that specifically bind to the antibodies or an antibody-deiminated peptide complex. In some embodiments, the secondary antibodies are labeled with a detectable label. In some embodiments, detecting antibodies comprises directly detecting an antibody-deiminated peptide complex attached to the solid support.

In some embodiments, the biological sample is selected from blood, cerebrospinal fluid (CSF), urine, saliva, stool, and synovial fluid. In some embodiments, the biological sample is blood.

In some embodiments, the subject is suspected of or at risk of having oxygen-deprivation brain injury (ODBI) or oxygen-deprivation causing injury (ODCI). In some embodiments, the subject is suspected of or at risk of having traumatic brain injury (TBI).

Further disclosed herein are methods of diagnosing ODBI or ODCI in a subject, comprising (a) analyzing a biological sample taken from the subject to detect antibodies specific to a deiminated variant of one or more of the proteins listed in Table 1, or a fragment thereof; and (b) comparing the detected antibody level(s) to reference antibody level(s) of antibodies specific to a deiminated variant of the same protein(s) in a reference sample, wherein an increase in the detected antibody level(s) as compared to the reference antibody level(s) is indicative that the subject is suffering from ODBI or ODCI. The methods optionally may further comprise detecting and comparing levels of antibodies specific to a deiminated variant of one or more proteins listed in Table 2 or a fragment thereof, optionally wherein the protein or fragment is deiminated at a site(s) listed in Table 2, and/or detecting and comparing levels of antibodies specific to a deiminated variant of one or more proteins listed in Table 3 or a fragment thereof, optionally wherein the protein or fragment is deiminated at a site(s) listed in Table 3.

Further disclosed herein are methods of diagnosing ODBI or ODCI in a subject, comprising (a) applying a biological sample taken from the subject to a device comprising a plurality of deiminated peptides to detect antibody level(s) in the biological sample, wherein at least one deiminated peptide comprises a deiminated portion of a deiminated variant of a protein listed in Table 1; and (b) comparing the detected antibody level(s) to reference antibody level(s) of antibodies specific to a deiminated variant of the same protein(s) in a reference sample, wherein an increase in the detected antibody level(s) as compared to the reference antibody level(s) is indicative that the subject is suffering from ODBI or ODCI. The device optionally may further comprise at least one deiminated peptide comprising a deiminated portion of a deiminated variant of a protein listed in Table 2 and/or a deiminated portion of a deiminated variant of a protein listed in Table 3.

In some embodiments, the reference sample is a sample previously obtained from the subject. Alternatively, in some instances, the reference sample is a sample from one or more reference subjects determined not to have ODBI or ODCI.

Further disclosed herein are methods of diagnosing ODBI or ODCI in a subject, comprising (a) analyzing a biological sample taken from the subject to detect antibodies specific to a deiminated variant of one or more of the proteins listed in Table 2 or a fragment thereof, wherein the deiminated variant is deiminated at a site(s) listed in Table 2; and (b) comparing the detected antibody level(s) to reference antibody level(s) of antibodies specific to a deiminated variant of the same protein(s) in a reference sample, wherein an increase in the detected antibody level(s) as compared to the reference antibody level(s) is indicative that the subject is suffering from ODBI or ODCI. The methods optionally may further comprise detecting and comparing levels of antibodies specific to a deiminated variant of one or more proteins listed in Table 1 or a fragment thereof, optionally wherein the protein or fragment is deiminated at a site(s) listed in Table 1, and/or detecting and comparing levels of antibodies specific to a deiminated variant of one or more proteins listed in Table 3 or a fragment thereof, optionally wherein the protein or fragment is deiminated at a site(s) listed in Table 3.

Further disclosed herein are methods diagnosing ODBI or ODCI in a subject, comprising (a) applying a biological sample taken from the subject to a device comprising a plurality of deiminated peptides to detect antibody level(s) in the biological sample, wherein at least one deiminated peptide comprises a deiminated portion of a deiminated variant of a protein listed in Table 2; and (b) comparing the detected antibody level(s) to reference antibody level(s) of antibodies specific to a deiminated variant of the same protein(s) in a reference sample, wherein an increase in the detected antibody level(s) as compared to the reference antibody level(s) is indicative that the subject is suffering from ODBI or ODCI. The device optionally may further comprise at least one deiminated peptide comprising a deiminated portion of a deiminated variant of a protein listed in Table 1 and/or a deiminated portion of a deiminated variant of a protein listed in Table 3.

In some embodiments, the reference sample is a sample previously obtained from the subject. Alternatively, in some embodiments, the reference sample is a sample from one or more reference subjects determined not to have ODBI or ODCI.

Further disclosed herein are methods of monitoring the progression of ODBI or ODCI in a subject, comprising: (a) analyzing at least two biological samples taken from the subject at different time points to detect level(s) of antibodies specific to a deiminated variant of one or more of the proteins listed in Table 1 or a fragment thereof; and (b) comparing the detected antibody level(s) in the biological samples taken at different time points, wherein an increase in the detected antibody level(s) over time is indicative that the subject's TBI status is increasing (worsening) over time, and/or wherein a decrease in the detected antibody level(s) over time is indicative that the subject's TBI status is decreasing (improving) over time. The methods optionally may further comprise detecting and comparing levels of antibodies specific to a deiminated variant of one or more proteins listed in Table 2 or a fragment thereof, optionally wherein the protein or fragment is deiminated at a site(s) listed in Table 2, and/or detecting and comparing levels of antibodies specific to a deiminated variant of one or more proteins listed in Table 3 or a fragment thereof, optionally wherein the protein or fragment is deiminated at a site(s) listed in Table 3.

Further disclosed herein are methods of monitoring the progression of ODBI or ODCI in a subject, comprising: (a) analyzing at least two biological samples taken from the subject at different time points to detect level(s) of antibodies specific to a deiminated variant of one or more of the proteins listed in Table 2 or a fragment thereof, wherein the deiminated variant is deiminated at a site(s) listed in Table 2; and (b) comparing the detected antibody level(s) in the biological samples taken at different time points, wherein an increase in the detected antibody level(s) over time is indicative that the subject's TBI status is increasing (worsening) over time, and/or wherein a decrease in the detected antibody level(s) over time is indicative that the subject's TBI status is decreasing (improving) over time. The methods optionally may further comprise detecting and comparing levels of antibodies specific to a deiminated variant of one or more proteins listed in Table 1 or a fragment thereof, optionally wherein the protein or fragment is deiminated at a site(s) listed in Table 1, and/or detecting and comparing levels of antibodies specific to a deiminated variant of one or more proteins listed in Table 3 or a fragment thereof, optionally wherein the protein or fragment is deiminated at a site(s) listed in Table 3.

Further disclosed herein are methods of monitoring the progression of ODBI or ODCI in a subject, comprising: (a) applying a first biological sample taken from the subject to a first device comprising a plurality of deiminated peptides to detect antibody level(s) in the first biological sample, wherein at least one deiminated peptide comprises a deiminated portion of a deiminated variant of a protein listed in Table 1; (b) applying a second biological sample taken from the subject to a second device comprising a plurality of deiminated peptides to detect antibody level(s) in the second biological sample, wherein at least one deiminated peptide comprises a deiminated portion of a deiminated variant of a protein listed in Table 1; and (c) comparing the detected antibody level(s) in the first and second biological samples, wherein an increase in the detected antibody level(s) over time is indicative that the therapy is effective, and/or wherein a decrease in the detected antibody level(s) over time is indicative that the therapy is effective. The devices optionally may further comprise at least one deiminated peptide comprising a deiminated portion of a deiminated variant of a protein listed in Table 2 and/or a deiminated portion of a deiminated variant of a protein listed in Table 3.

Further disclosed herein are methods of monitoring the progression of ODBI or ODCI in a subject, comprising: (a) applying a first biological sample taken from the subject to a first device comprising a plurality of deiminated peptides to detect antibody level(s) in the first biological sample, wherein at least one deiminated peptide comprises a deiminated portion of a deiminated variant of a protein listed in Table 2; (b) applying a second biological sample taken from the subject to a second device comprising a plurality of deiminated peptides to detect antibody level(s) in the second biological sample, wherein at least one deiminated peptide comprises a deiminated portion of a deiminated variant of a protein listed in Table 2; and (c) comparing the detected antibody level(s) in the first and second biological samples, wherein an increase in the detected antibody level(s) over time is indicative that the therapy is effective, and/or wherein a decrease in the detected antibody level(s) over time is indicative that the therapy is effective. The device optionally may further comprise at least one deiminated peptide comprising a deiminated portion of a deiminated variant of a protein listed in Table 1 and/or a deiminated portion of a deiminated variant of a protein listed in Table 3.

Further disclosed herein are methods of determining the efficacy of an ODBI-therapy or ODCI-therapy in a subject treated with the ODBI-therapy or ODCI-therapy, comprising: (a) analyzing at least two biological samples taken from the subject at different time points to detect level(s) of antibodies specific to a deiminated variant of one or more of the proteins listed in Table 1 or a fragment thereof; and (b) comparing the detected antibody level(s) in the biological samples taken at different time points, wherein an increase in the detected antibody level(s) over time is indicative that the therapy is not effective, and/or wherein a decrease in the detected antibody level(s) over time is indicative that the therapy is effective. The methods optionally may further comprise detecting and comparing levels of antibodies specific to a deiminated variant of one or more proteins listed in Table 2 or a fragment thereof, optionally wherein the protein or fragment is deiminated at a site(s) listed in Table 2, and/or detecting and comparing levels of antibodies specific to a deiminated variant of one or more proteins listed in Table 3 or a fragment thereof, optionally wherein the protein or fragment is deiminated at a site(s) listed in Table 3.

Further disclosed herein are methods of determining the efficacy of an ODBI-therapy or ODCI-therapy in a subject treated with the ODBI-therapy or ODCI-therapy, comprising: (a) analyzing at least two biological samples taken from the subject at different time points to detect level(s) of antibodies specific to a deiminated variant of one or more of the proteins listed in Table 2 or a fragment thereof, wherein the deiminated variant is deiminated at a site(s) listed in Table 2; and (b) comparing the detected antibody level(s) in the biological samples taken at different time points, wherein an increase in the detected antibody level(s) over time is indicative that the therapy is not effective, and/or wherein a decrease in the detected antibody level(s) over time is indicative that the therapy is effective. The methods optionally may further comprise detecting and comparing levels of antibodies specific to a deiminated variant of one or more proteins listed in Table 1 or a fragment thereof, optionally wherein the protein or fragment is deiminated at a site(s) listed in Table 1, and/or detecting and comparing levels of antibodies specific to a deiminated variant of one or more proteins listed in Table 3 or a fragment thereof, optionally wherein the protein or fragment is deiminated at a site(s) listed in Table 3.

Further disclosed herein are methods of determining the efficacy of an ODBI-therapy or ODCI-therapy in a subject treated with the ODBI-therapy or ODCI-therapy, comprising: (a) applying a first biological sample taken from the subject to a first device comprising a plurality of deiminated peptides to detect antibody level(s) in the first biological sample, wherein at least one deiminated peptide comprises a deiminated portion of a deiminated variant of a protein listed in Table 1; (b) applying a second biological sample taken from the subject to a second device comprising a plurality of deiminated peptides to detect antibody level(s) in the second biological sample, wherein at least one deiminated peptide comprises a deiminated portion of a deiminated variant of a protein listed in Table 1; and(c) comparing the detected antibody level(s) in the first and second biological samples, wherein an increase in the detected antibody level(s) over time is indicative that the therapy is effective, and/or wherein a decrease in the detected antibody level(s) over time is indicative that the therapy is effective. The devices optionally may further comprise at least one deiminated peptide comprising a deiminated portion of a deiminated variant of a protein listed in Table 2 and/or a deiminated portion of a deiminated variant of a protein listed in Table 3.

Further disclosed herein are methods of determining the efficacy of an ODBI-therapy or ODCI-therapy in a subject treated with the ODBI-therapy or ODCI-therapy, comprising: (a) applying a first biological sample taken from the subject to a first device comprising a plurality of deiminated peptides to detect antibody level(s) in the first biological sample, wherein at least one deiminated peptide comprises a deiminated portion of a deiminated variant of a protein listed in Table 2; (b) applying a second biological sample taken from the subject to a second device comprising a plurality of deiminated peptides to detect antibody level(s) in the second biological sample, wherein at least one deiminated peptide comprises a deiminated portion of a deiminated variant of a protein listed in Table 2; and(c) comparing the detected antibody level(s) in the first and second biological samples, wherein an increase in the detected antibody level(s) over time is indicative that the therapy is effective, and/or wherein a decrease in the detected antibody level(s) over time is indicative that the therapy is effective. The devices optionally may further comprise at least one deiminated peptide comprising a deiminated portion of a deiminated variant of a protein listed in Table 1 and/or a deiminated portion of a deiminated variant of a protein listed in Table 3.

In some embodiments, the subject is administered the ODBI-therapy or ODCI-therapy prior to the second biological sample is taken from the subject.

In some embodiments, the ODBI-therapy and/or ODCI-therapy comprises one or more anti-immune drugs and/or anti-idiotypic antibodies.

In some embodiments, the antibodies are detected by contacting the biological sample(s) with a deiminated peptide that comprises at least a deiminated portion of the deiminated variant. In some embodiments, the deiminated peptide is deiminated at a listed site(s). In some embodiments, the deiminated peptide comprise from 3 to 25 amino acid residues. In some embodiments, the deiminated peptide is attached to a solid support.

In some embodiments, the antibodies are detected by contacting any antibodies bound to the deiminated peptide with secondary antibodies that specifically bind to the antibodies or an antibody-deiminated peptide complex. In some embodiments, the secondary antibodies are labeled with a detectable label. In some embodiments, the antibodies are detected by directly detecting the antibody-deiminated peptide complex attached to the solid support.

In some embodiments, the biological sample(s) is selected from blood, cerebrospinal fluid (C SF), urine, saliva, stool, and synovial fluid. In some embodiments, the biological sample is blood.

Also disclosed herein are devices comprising a plurality of deiminated peptides, wherein at least one deiminated peptide comprises a deiminated portion of a deiminated variant of a protein listed in Table 1. In some embodiments, the devices disclosed herein further comprise one or more deiminated peptides comprising a deiminated portion of a deiminated variant of a protein listed in Table 2 and/or Table 3 or a fragment thereof

Further disclosed herein are devices comprising a plurality of deiminated peptides, wherein at least one deiminated peptide comprises a deiminated portion of a deiminated variant of a protein listed in Table 2 that is deiminated at a site listed in Table 2. In some embodiments, the devices disclosed herein further comprise one or more deiminated peptides comprising a deiminated portion of a deiminated variant of a protein listed in Table 3 or a fragment thereof.

In some embodiments, the devices disclosed herein comprise one or more deiminated peptides comprising a deiminated portion of a deiminated variant of a protein listed in Tables 1-3 or a fragment thereof.

In some embodiments, the deiminated peptides are attached to a solid support.

Further disclosed herein are methods for treating an ODBI or ODCI in a subject in need thereof, wherein the subject may be identified as being in need of treatment by the methods disclosed herein. In some embodiments, such methods comprise administering to the subject a deiminase inhibitor. In some embodiments, such methods comprise administering to the subject an agent that inhibits an immune response to a deiminated protein as disclosed herein, such as an agent that inhibits an antibody specific to a deiminated variant of one or more of the deiminated proteins disclosed herein, such as an antibody that is anti-idotypic to an antibody specific to a deiminated variant of one or more of the deiminated proteins disclosed herein.

In some embodiments, the deiminase inhibitor is a reversible inhibitor. In some embodiments, the deiminase inhibitor is an irreversible inhibitor. In some embodiments, the deiminase inhibitor inhibits or suppresses the activity of a protein arginine deiminase (PAD). In some embodiments, the PAD is selected from PAD 1, PAD 2, PAD 3, PAD 4, and PAD 6. In some embodiments, the deiminase inhibitor is selected from taxol, minocycline, streptomycin, o-F-amidine, o-Cl-amidine, Thr-Asp-F-amidine (TDFA), thioredoxin, streptonigrin, and an analog or derivative thereof. In some embodiments, the deiminase inhibitor inhibits or suppresses the deimination of one or more proteins listed in Table 1, Table 2, or Table 3.

In some embodiments, the method for treating an ODBI or ODCI in a subject in need thereof comprises administering to the subject an agent that inhibits an antibody specific to a deiminated variant of one or more of the proteins listed in Table 1 (or a fragment thereof). In some embodiments, the method for treating an ODBI or ODCI in a subject in need thereof comprises administering to the subject an agent that inhibits an antibody specific to a deiminated variant that is deiminated at the site(s) listed in Table 1. In some embodiments, the method for treating an ODBI or ODCI in a subject in need thereof comprises administering to the subject an agent that inhibits an antibody specific to a deiminated variant of one or more of the proteins listed in Table 2 (or a fragment thereof). In some embodiments, the method for treating an ODBI or ODCI in a subject in need thereof comprises administering to the subject an agent that inhibits an antibody specific to a deiminated variant that is deiminated at the site(s) listed in Table 2. In some embodiments, the method for treating an ODBI or ODCI in a subject in need thereof comprises administering to the subject an agent that inhibits an antibody specific to a deiminated variant of one or more of the proteins listed in Table 3 (or a fragment thereof). In some embodiments, the method for treating an ODBI or ODCI in a subject in need thereof comprises administering to the subject an agent that inhibits an antibody specific to a deiminated variant that is deiminated at the site(s) listed in Table 3. In any embodiments, the agent may be an antibody that is anti-idotypic to an antibody specific to a deiminated variant as disclosed herein.

Further disclosed herein is a method for treating an ODBI or ODCI in a subject, comprising: (a) analyzing a biological sample taken from the subject to detect antibodies specific to one or more of a deiminated variant of (i) one or more of the proteins listed in Table 1 (or a fragment thereof); (ii) one or more protein listed in Table 2 (or a fragment thereof); and/or (iii) one or more proteins listed in Table 3 (or a fragment thereof); (b) comparing the detected antibody level(s) to reference antibody level(s) of antibodies specific to a deiminated variant of the same protein(s) in a reference sample, wherein an increase in the detected antibody level(s) as compared to the reference antibody level(s) is indicative that the subject is suffering from ODBI or ODCI; and (c) administering to a subject indicated to be suffering from ODBI or ODCI a deiminase inhibitor, optionally wherein the deiminase inhibitor inhibits or suppresses the activity of a protein arginine deiminase (PAD), optionally wherein the PAD is selected from PAD 1, PAD 2, PAD 3, PAD 4, and PAD 6, optionally wherein the deiminase inhibitor is selected from taxol, minocycline, streptomycin, o-F-amidine, o-Cl-amidine, Thr-Asp-F-amidine (TDFA), thioredoxin, streptonigrin, and analogs and derivatives thereof.

Further disclosed herein is a method for treating an ODBI or ODCI in a subject, comprising: administering a deiminase inhibitor to a subject determined to have elevated levels of antibodies specific to one or more of a deiminated variant of (i) one or more of the proteins listed in Table 1 (or a fragment thereof); (ii) one or more protein listed in Table 2 (or a fragment thereof); and/or (iii) one or more proteins listed in Table 3 (or a fragment thereof), as compared to reference antibody levels, optionally wherein the deiminase inhibitor inhibits or suppresses the activity of a protein arginine deiminase (PAD), optionally wherein the PAD is selected from PAD 1, PAD 2, PAD 3, PAD 4, and PAD 6, optionally wherein the deiminase inhibitor is selected from taxol, minocycline, streptomycin, o-F-amidine, o-Cl-amidine, Thr-Asp-F-amidine (TDFA), thioredoxin, streptonigrin, and analogs and derivatives thereof.

Further disclosed herein is a method for treating an ODBI or ODCI in a subject, comprising: (a) analyzing a biological sample taken from the subject to detect antibodies specific to one or more of a deiminated variant of (i) one or more of the proteins listed in Table 1 (or a fragment thereof); (ii) one or more protein listed in Table 2 (or a fragment thereof); and/or (iii) one or more proteins listed in Table 3 (or a fragment thereof); (b) comparing the detected antibody level(s) to reference antibody level(s) of antibodies specific to a deiminated variant of the same protein(s) in a reference sample, wherein an increase in the detected antibody level(s) as compared to the reference antibody level(s) is indicative that the subject is suffering from ODBI or ODCI; and (c) administering to a subject indicated to be suffering from ODBI or ODCI an agent that inhibits an antibody specific to a deiminated variant of (i) one or more of the proteins listed in Table 1 (or a fragment thereof), optionally that inhibits an antibody specific to a deiminated variant that is deiminated at the site(s) listed in Table 1; (ii) one or more of the proteins listed in Table 2 (or a fragment thereof), optionally that inhibits an antibody specific to a deiminated variant that is deiminated at the site(s) listed in Table 2; and/or (iii) one or more of the proteins listed in Table 3 (or a fragment thereof), optionally that inhibits an antibody specific to a deiminated variant that is deiminated at the site(s) listed in Table 3, optionally wherein the agent is an anti-idiotypic antibody idiotypic to the antibody specific to the deiminated variant.

Further disclosed herein is a method for treating an ODBI or ODCI in a subject, comprising: administering an agent to a subject determined to have elevated levels of antibodies specific to one or more of a deiminated variant of (i) one or more of the proteins listed in Table 1 (or a fragment thereof); (ii) one or more protein listed in Table 2 (or a fragment thereof); and/or (iii) one or more proteins listed in Table 3 (or a fragment thereof), as compared to reference antibody levels, wherein the agent inhibits an antibody specific to a deiminated variant of (i) one or more of the proteins listed in Table 1 (or a fragment thereof), optionally that inhibits an antibody specific to a deiminated variant that is deiminated at the site(s) listed in Table 1; (ii) one or more of the proteins listed in Table 2 (or a fragment thereof), optionally that inhibits an antibody specific to a deiminated variant that is deiminated at the site(s) listed in Table 2; and/or (iii) one or more of the proteins listed in Table 3 (or a fragment thereof), optionally that inhibits an antibody specific to a deiminated variant that is deiminated at the site(s) listed in Table 3, optionally wherein the agent is an anti-idiotypic antibody idiotypic to the antibody specific to the deiminated variant.

Any treatment methods may further comprise monitoring the progression of ODBI or ODCI in the subject, monitoring the treatment of ODBI or ODCI in the subject, and/or monitoring the efficacy of the treatment of ODBI or ODCI in the subject, as described herein.

### BRIEF DESCRIPTION OF THE DRAWINGS

FIG. 1 illustrates protein deimination which is catalyzed by a family of structurally-related, calcium-dependent enzymes known as peptidylarginine deiminases (PADs). Protein deimination involves the conversion of an intra-protein arginine residue to a citrulline residue, resulting in the loss of a positively charged amine group and an increase of 1 Da in molecular mass.

FIGS. 2A-2B illustrates detection of blast-induced deiminated proteins in porcine brain. Brain samples were collected 2 weeks following a single blast exposure (average pressure=46 psi). Homogenates of control (C) and blast-exposed (B) cerebral cortex were pre-fractionated by liquid phase isoelectric focusing (LP-IEF_. The resulting pH fractions were further fractionated by (1-dimensional sodium dodecyl sulfate polyacrylamide gel electrophoresis (SDS-PAGE) and analyzed for protein deimination by western blotting using the mouse monoclonal 6B3 anti-protein citrulline antibody, as show in FIG. 2B. Immunoreactive features affected by TBI (numbered in FIG. 2B) were mapped to corresponding bands in a Coomassie stained protein gel, as shown in FIG. 2A. These bands were collected, identified and mapped for site-specific deimination by peptide mass fingerprinting using liquid chromatography and tandem mass spectrometry (LC MS/MS).

FIG. 3 illustrates mapping of protein deimination sites by neutral loss. Tryptic peptides were fragmented by collision-induced dissociation and resulting spectra analyzed for a neutral loss of 43 Da, reflecting the loss of isocyanic acid as a fragmentation product of citrulline (upper panel). The representative spectrum shown depicts the Y and B ion spectra of GFAP peptide, TVEMrDGEVIK (SEQ ID NO: 146), with the neutral loss peak observed for the deiminated arginine (r) at 625.8 Da. Because the parent peptide ion was doubly charged in this case, the observed neutral loss in the spectrum was 21.5 Da, reflecting 43 Da/2.

FIGS. 4A-C show the effects of blast exposure on the presence of IgG expression in the cerebral cortex of swine. Homogenates of control, and blast-exposed (2 weeks post-injury) cerebral cortex (N=4/group) were fractionated by 1-dimensional SDS-PAGE (FIG. 4A) and analyzed for IgG content by Western blotting (FIG. 4B). Immunoreactive heavy (H) and light (L) chain IgGs were visualized using an anti-porcine IgG detection antibody. The values for the total IgG chemiluminescence signal (H+L) (FIG. 4C) for each sample was standardized to protein load (A) by densitometry analysis using ImageJ, and resulting values were analyzed statistically. The relative signal intensity is shown on the Y-axis as densitometry values ×100. Data are presented as the mean+standard error; *p≤0.05.

FIG. 5 presents a proposed mechanism for the role of aberrant protein deimination in an autoimmune response to brain injury. TBI-induced calcium excitotoxicity hyper-activates PAD resulting in an abnormal pattern of protein deimination. Cells of the adaptive immune system process the antigenic epitopes created by deimination. Antigen presentation and T-cell activation subsequently lead to the activation of B-cells for the production of autoantibodies and chronic neuroinflammation. It is proposed that these mechanisms contribute to long-term pathologies that can result from TBI. Potential therapeutic interventions that inhibit protein deimination and T-cell and B-cell activation are depicted with red lines.

FIGS. 6A-6D provide schematics of a deiminated peptide (FIGS. 6A and 6B), a microarray comprising deiminated peptides (FIG. 6C), and a spherical particle comprising deiminated peptides (FIG. 6D), relating to devices described herein.

FIG. 7 provides a schematic of an exemplary assay for detecting antibodies specific to deiminated proteins.

FIG. 8 provides a schematic for designing deiminated peptides useful for detecting anti-deimination antibodies as described herein.

### DETAILED DESCRIPTION

Described herein are specific brain proteins that are deiminated in response to oxygen deprivation. The deiminated proteins display antigenic epitopes that are recognized by the immune system, producing an autoimmune response that includes the generation of autoantibodies that specifically bind to epitopes of the deiminated proteins. These autoantibodies can be detected and/or measured in biological samples, such as blood. Thus, methods of detecting these autoantibodies also are described. Also described are devices for detecting one or more autoantibodies specific for the deiminated proteins described herein, including multiplex platforms capable of detecting a plurality of different autoantibodies. The detection of autoantibodies to the deiminated proteins described herein allows for the detection, diagnosis and/or treatment of ODBI and ODCI, including TBI.

### DEFINITIONS

Technical and scientific terms used herein have the meanings commonly understood by one of ordinary skill in the art to which the present disclosure pertains, unless otherwise defined.

As used herein, the singular forms “a,” “an,” and “the” designate both the singular and the plural, unless expressly stated to designate the singular only.

As used herein, “subject” denotes any mammal, including humans. For example, a subject may be suspected of having ODBI or ODCI, including TBI, at risk of ODBI or ODCI, including TBI, or suffering from ODBI or ODCI, including TBI, such as having been exposed to, or possibly exposed to, a force or impact capable of causing ODBI or ODCI, such as TBI.

The terms “administer,” “administration,” or “administering” as used herein refer to (1) providing, giving, dosing and/or prescribing, such as by either a health professional or his or her authorized agent or under his direction, and (2) putting into, taking or consuming, such as by a health professional or the subject.

The terms “treat”, “treating” or “treatment”, as used herein, include alleviating, abating or ameliorating a disease or condition or one or more symptoms thereof, whether or not the disease or condition is considered to be “cured” or “healed” and whether or not all symptoms are resolved. The terms also include reducing or preventing progression of a disease or condition or one or more symptoms thereof, impeding or preventing an underlying mechanism of a disease or condition or one or more symptoms thereof, and achieving any therapeutic and/or prophylactic benefit.

The terms “specific to” or “specific for” as used herein in the context of an antibody or antibodies refer to the preferential binding of an antibody to an antigen or epitope. The preferential binding of an antibody to an antigen or epitope may be based on the affinity and/or specificity of the antibody for the antigen or epitope. Antibody affinity refers to the strength of the interaction between an antibody and its respective epitope. Antibody specificity refers to the degree to which the antibody discriminates between antigenic variants. For instance, the phrase “an antibody specific to a deiminated variant of a protein” means the antibody preferentially binds an epitope characteristic of the deiminated variant of a protein as compared to an epitope of a different deiminated variant of the same protein, and/or as compared to an epitope of a non-deiminated variant of the same protein, and/or as compared to an epitope of another deiminated or non-deiminated protein. As used herein, an antibody specific to a deiminated variant is distinct from an anti-deimination antibody, which only reacts with a deiminated arginine residue in any protein, and from other anti-protein antibodies that bind to an epitope that is characteristic of a non-deiminated protein.

As used herein, the term “deiminated variant” refers to a deiminated version of the protein, i.e., a variant of the protein wherein one or more of the arginine residues is/are deiminated.

The term “epitope characteristic of a deiminated variant” as used herein refers to a region within the deiminated variant that is characteristic of the deiminated variant as compared to a different deiminated variant and as compared to the non-deiminated variant, and that is recognized (bound) by an antibody. An epitope characteristic of a deiminated variant may or may not comprise the deiminated residue. For example, a deiminated variant may have a different three-dimensional structure that presents one or more epitopes that are not presented by a different deiminated variant or by the non-deiminated variant, such as may arise from altered protein folding. Accordingly, in some instances, an epitope characteristic of a deiminated variant comprises a region within the deiminated protein having a tertiary structure that is characteristic of the deiminated protein (e.g., a tertiary structure that results from a different protein folding in deiminated protein that is not present in the non-deiminated protein).

As used herein, the term “oxygen-deprivation brain injury” (ODBI) refers to a brain injury that results from oxygen deprivation to the brain. Examples of ODBIs include, but are not limited to, hypoxic brain injury and anoxic brain injury. Hypoxia (e.g., reduced oxygen to the brain) may cause long-term disabilities and developmental delays. Anoxia (e.g., complete deprivation of oxygen to the brain) can cause similar disabilities and death, if the anoxia is prolonged.

As used herein, the term “oxygen-deprivation causing injury” (ODCI) refers to an injury, disease or condition that causes oxygen deprivation to the brain, which may lead to an oxygen deprivation brain injury. Examples of ODCIs include, but are not limited to, traumatic brain injury (TBI), stroke, hemorrhage, suffocation, drowning, choking, strangulation, seizure, intoxication, smoke inhalation, cardiac arrest, complications at birth, and cardiopulmonary resuscitation (CPR). Examples of complications at birth include trauma to the infant in utero, problems with the placenta, umbilical cord prolapsed, preeclampsia, eclampsia, and hemorrhage.

As used herein, the term “ODBI-therapy” refers to a therapy that is used treat an ODBI. For instance, if the ODBI is a hypoxic injury, then the ODBI-therapy is a therapy that is used to treat a hypoxic injury, such as supplemental oxygen therapy. The ODBI-therapy also includes rehabilitative therapy used to treat an ODBI.

As used herein, the term “ODCI-therapy” refers to a therapy that is used to treat an ODCI. For instance, if the ODCI is a hemorrhage, then the ODCI-therapy is a therapy that is used to treat a hemorrhage, such as anti-anxiety or anti-epileptic drugs. The ODCI-therapy also includes rehabilitative therapy used to treat an ODCI.

In some embodiments, the methods disclosed herein relate to detecting specific biomarkers of ODBI or OCDI, such as deiminated proteins or autoantibodies thereto or levels of IgG in a biological sample from a subject. In some embodiments, the ODCI is TBI. The TBI may be any TBI, including mild TBI (mTBI) or severe TBI (sTBI), closed head injury (CHI) or blast-induced traumatic brain injury (bTBI). The TBI may be a concussion, contusion, coup-contrecoup injury, diffuse axonal injury, or penetration injury. In accordance with specific embodiments, the TBI is blast-induced traumatic brain injury (bTBI). In accordance with specific embodiments of any of the methods described herein, the subject may be a human.

### Deiminated Protein and Oxygen Deprivation

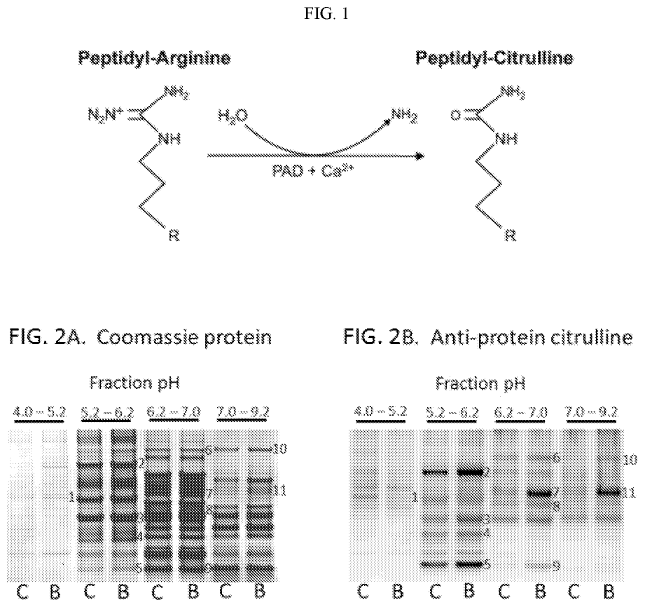

Deimination, also referred to as citrullination, is a posttranslational modification involving the calcium-dependent conversion of peptidyl-arginine to peptidyl-citrulline catalyzed by peptidylarginine deiminase (PAD) (FIG. 1). This modification can result in the creation of novel, potentially antigenic epitopes that can elicit autoimmune responses [19, 20] (FIG. 1). For example, disordered deimination of the joint proteins, filaggrin [21] and vimentin [22], generates antigenic epitopes [23] which can trigger a sustained autoimmune attack that eventually destroys the synovial compartment [24]. Disorders in protein deimination are also implicated in diseases of the central nervous system, most notably multiple sclerosis [25-27], where the deimination of myelin basic protein appears to underlie a sustained autoimmune attack against the deiminated protein [28]. Abnormal protein deimination is also an underlying mechanism in the autoimmune diseases, such as rheumatoid arthritis (RA), where aberrant deimination creates antigenic epitopes that elicit sustained autoimmune attacks.

There is increasing interest in the possibility that the immune system plays a role in the long-term pathogenesis of TBI [29, 30]. Oxidative stress and calcium excitotoxicity are hallmarks of TBI, which create a cellular environment that promotes aberrant protein deimination. Protein deimination is mediated by a small family of calcium-dependent enzymes, peptidylarginine deiminase (PAD), which is expressed in a tissue specific and developmentally regulated fashion and exhibits a selective subcellular localization. While most is known about the role of PAD in the nucleus, where it is involved in epigenetic regulation of gene expression via the deimination of histones, PAD is also present in the cytosol where its functions are not well understood. It was previously reported that controlled cortical impact in rodents selectively alters the deimination status of a subset of proteins constituting the brain proteome [31], presumably due to injury-induced conditions of oxidative stress and calcium excitotoxicity.

As set forth in the examples below, a large animal model (porcine) study using blast injury as a non-invasive form of TBI showed that only a small subset of the entire brain proteome underwent blast-induced deimination in porcine brain. Six of the proteins involved were identified (see Table 4). Two of these were vimentin and glial fibrillary acidic protein (GFAP). The deimination sites found within vimentin and GFAP corresponded to previously reported sites of deimination in rheumatoid arthritis for vimentin and in multiple sclerosis [32] and Alzheimer's disease [33, 34] for GFAP.

As also set forth in the examples below, a study was conducted in humans using brain tissue from human subjects who experienced motor vehicle accidents resulting in acute fatal trauma, and suffered oxygen deprivation to the brain. This study examined the status of protein deimination in the cerebral cortex. This study has identified a human ODBI deiminome (a collection of proteins that are found to be deiminated in subjects suffering from oxygen deprivation to the brain) that can now serve as the basis for autoimmune profiling in ODBI and/or ODCI. A total of 90 different proteins with 147 distinct deimination sites were identified (see Tables 1-3). Based upon function, structural proteins and mitochondrial enzymes were most highly represented. The most heavily deiminated protein identified, with eight sites, was 2′3′-cyclic-nucleotide 3′-phosphodiesterase; a myelin-associated enzyme that makes up 4% of total myelin protein. Fructose biphosphate aldolase C was found to possess seven deimination sites.

A number of the proteins identified are established autoantigens (e.g., myelin basic protein (MBP), glial fibrillary acidic proteins (GFAP), collapsin response mediator protein-2 (CRMP2, dihydropyrimidinase-related protein 2) gamma enolase, 78 kDa glucose-regulated protein, moesin, microtubule-associated protein tau, vimentin and contactin). However, many of the proteins were newly identified autoantigens (Table 1) and the deimination sites reported in Tables 1-2 are newly identified. Wang and coworkers [39] have reported the expression of anti-GFAP autoantibodies in humans following brain injury. Similar findings were reported for the brain protein, S100b [43].

While not wanting to be bound by theory, each identified deiminated arginine residue identified here represents a potential epitope for immune recognition and autoimmune-based neuropathology; thus, immune reactions to one or more of these epitopes may contribute to the impaired cognition and mood imbalances that can persist for years post-ODBI and/or post-ODCI, such as post-TBI, even in the absence of any evidence of overt injury based upon findings employing the most advanced neuroimaging technologies. Autoimmune profiling may, therefore, be useful in detecting autoantibodies to proteins modified by aberrant deimination, and thus serve to diagnose and monitor ODBI and/or ODCI, such as TBI, and guide the use of immune therapies for treating chronic consequences of ODBI and/or ODCI. Additionally, these findings will support the use of acute treatments designed to inhibit aberrant protein deimination (e.g., use of small molecule inhibitors of PAD) immediately following ODBI and/or ODCI, such as TBI.

Thus, in one aspect, the invention described herein relates to the detection of one or more autoantibodies targeting the deiminated proteins described, herein, as biomarkers for understanding and assessing ODBI and/or ODCI pathogenesis, including TBI pathogenesis.

As noted in the examples below, the porcine study also determined that levels of immunoglobulin G (IgG) detected in the brains of blast-exposed animals were markedly elevated as compared to those present in control animals, possibly representing autoantibodies directed against novel protein epitopes. Thus, in another aspect, the invention relates to the use of a biological sample, such as cerebral spinal fluid (CSF) or blood sample, to detect IgG levels as biomarkers for ODBI and/or ODCI (such as TBI).

Together, these findings indicate that autoantibodies, resulting from ODBI and ODCI aberrant protein deimination, may underlie chronic neuropathologies associated with ODBI and ODCI through mechanisms involving the adaptive immune system.

As noted above, brain injury can result in long-term symptomologies that include impaired learning and memory, poor concentration/attention, slowed thinking, emotional and mood imbalances including increased anxiety, depression, disorientation, headaches, and emotional and cognitive dysfunction. These problems can persist for years after injury, often in the absence of any detectable neuropathology [14, 15]. At the cellular level, however, brain injury can result in a sustained state of neuroinflammation that is reflected in the proinflammatory, Ml phenotype of microglia [41, 42]. The persistence of these responses is consistent with the involvement of the adaptive immune system [29, 30]. The findings described herein raise the possibility that aberrant deimination of specific brain proteins and the immune response thereto may be an important mechanism in this phenomenon.

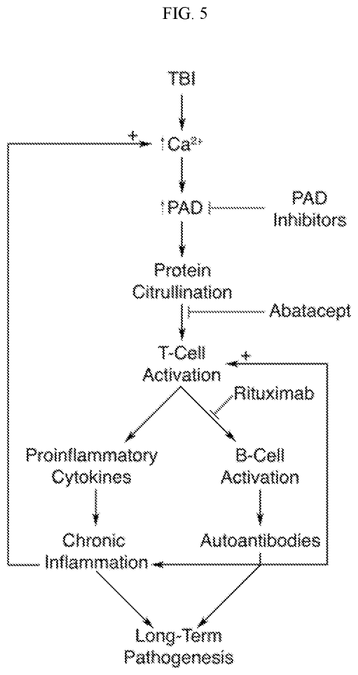

A working model for the sequence of events that could result in a brain-specific autoimmune response to ODBI and/or ODCI is presented in FIG. 5. Central to this model are: injury-induced oxidative stress and calcium excitotoxicity, hyperactivation of PADs, aberrant protein deimination, T-and B-cell activation in response to novel deiminated autoantigens, and the resulting establishment of a chronic inflammatory state via sustained activation of the adaptive immune system. This potential mechanism involving the adaptive immune system presents a substantial concern for long-term pathogenesis, as well as a target for therapy.

Also depicted in the model are three avenues for therapeutic intervention that address the inhibition of PAD, as well as T- and B-cell activation. To date, PAD inhibitors have not been tested in humans, although one or more prototype drugs are expected to reach clinical trials in the fall of 2018. Recent evidence indicates that the therapeutic effectiveness of the autoimmune therapies, abatacept [44] and rituximab [45, 46], in rheumatoid arthritis is directly related to the titer of autoantibodies reactive to deiminated proteins in the synovial compartment. Accordingly, models of long-term brain injury involving an autoimmune response to deiminated proteins may benefit from these therapies. Thus, in one aspect, the invention informs the use of PAD inhibitors, abatacept, or rituximab, in the treatment of ODBI and/or ODCI (such as TBI). In another aspect, the invention informs the use of anti-idiotypic antibodies idiotypic to an antibody specific to one or more deiminated proteins described herein, for the treatment of ODBI and/or ODCI (such as TBI).

The relations between protein deimination and the adaptive immune system in rheumatoid arthritis and neurodegenerative diseases suggest that these states may share a common mechanism. First, as reported for rheumatoid arthritis, multiple sclerosis [47], Alzheimer's disease [48] and prion disease [49] have distinctive profiles of protein deimination. Second, the vimentin sequence of TVETrDGQVINETSQHHDDLE (SEQ ID NO: 149) identified here in blast injury precisely matches the site (indicated by “r”) found to be deiminated in rheumatoid arthritis [40]. Third, the deimination site in GFAP observed here, TVEMrDGEVIK (SEQ ID NO: 146), was reported as a deimination site in Alzheimer's disease [33, 34]. And four, the core sequence of this peptide, EMrDGEVIK (SEQ ID NO: 151), has also been shown to be reactive with circulating autoantibodies in a patient with relapsing-remittent multiple sclerosis [32]. On the basis of these findings, it is proposed that aberrant protein deimination and subsequent involvement of the adaptive immune system may be an underlying mechanism shared by chronic neurodegenerative diseases and classical autoimmune diseases.

In summary, the examples demonstrate that ODBI affects the deimination status of select brain proteins and also is associated with an increase in IgG levels in the brain. These findings provide the basis for a mechanistic link between the acute processes of ODBI and the expression of sustained neuropathology involving activation of the adaptive immune system. Thus, we propose a role for the adaptive immune system in mediating chronic pathologies of ODBI. Brain injury establishes a cellular environment that promotes aberrant protein deimination via activation of the calcium-dependent enzymes involved. The deimination modification can generate antigenic epitopes for activation of a sustained autoimmune response.

### Detection and Diagnostic Methods

Disclosed herein are methods for indirectly detecting deiminated proteins described herein. In specific embodiments, the methods involve detecting antibodies specific to the deiminated proteins described herein, such as autoantibodies present in a biological sample obtained from the subject. Generally, the methods involve analyzing a biological sample obtained from a subject and detecting antibodies present in the biological sample, wherein the antibodies are specific to a deiminated variant of one or more of the proteins described herein (or a fragment thereof). As noted above, as used herein, the term “deiminated variant” refers to a deiminated version of the protein, i.e., a variant of the protein wherein one or more of the arginine residues is/are deiminated.

Also disclosed herein are methods of diagnosing the consequences of ODBI or ODCI in a subject. Generally, the diagnostic methods comprise (a) analyzing a biological sample taken from the subject to detect antibodies specific to a deiminated variant of one or more of the proteins described herein (or a fragment thereof) and (b) comparing the detected antibody level(s) to level(s) of antibodies specific to a deiminated variant of the same protein(s) in a reference sample, wherein an increase in the detected antibody level(s) as compared to the reference antibody level(s) is indicative that the subject is suffering from ODBI or ODCI and their consequences. In some embodiments, the reference antibody level(s) are levels of antibodies specific to the same deiminated variant(s) as the detected antibody level(s). In some embodiments, the reference antibody level(s) are levels of antibodies specific to different deiminated variant(s) of the same protein(s). In some embodiments, the reference antibody level(s) are levels of antibodies specific to the same epitopes of the same deiminated variant(s) as the detected antibody level(s). In some embodiments, the reference antibody level(s) are levels of antibodies specific to different epitopes of the same deiminated variant(s) as the detected antibody level(s).

Also disclosed herein are methods of diagnosing ODBI or ODCI in a subject, comprising: (a) applying a biological sample taken from the subject to a device comprising a plurality of deiminated peptides to detect antibody level(s) in the biological sample, wherein at least one deiminated peptide comprises a deiminated portion of a deiminated variant of a protein listed in Table 1; and (b) comparing the detected antibody level(s) to reference antibody level(s) of antibodies specific to a deiminated variant of the same protein(s) in a reference sample, wherein an increase in the detected antibody level(s) as compared to the reference antibody level(s) is indicative that the subject is suffering from ODBI or ODCI.

Also disclosed herein are methods of diagnosing ODBI or ODCI in a subject, comprising: (a) applying a biological sample taken from the subject to a device comprising a plurality of deiminated peptides to detect antibody level(s) in the biological sample, wherein at least one deiminated peptide comprises a deiminated portion of a deiminated variant of a protein listed in Table 2; and (b) comparing the detected antibody level(s) to reference antibody level(s) of antibodies specific to a deiminated variant of the same protein(s) in a reference sample, wherein an increase in the detected antibody level(s) as compared to the reference antibody level(s) is indicative that the subject is suffering from ODBI or ODCI.

In some embodiments, the reference sample is a sample previously obtained from the same subject, such as before the subject incurred ODBI or ODCI, such as a sample previously obtained from the same subject prior to being exposed to a risk of incurring ODBI or ODCI. In some embodiments, the reference sample is a sample from one or more reference subjects determined not to have ODBI or ODCI.

Also disclosed herein are methods of monitoring the progression or treatment of ODBI or ODCI in a subject. In some embodiments, the method comprises (a) analyzing at least two biological samples taken from the subject at different time points to detect level(s) of antibodies specific to a deiminated variant of one or more of the proteins described herein (or a fragment thereof) and (b) comparing the detected antibody level(s) in the biological samples taken at different time points, wherein an increase in the detected antibody level(s) over time is indicative that the subject's ODBI or ODCI status is increasing (worsening) over time, and/or wherein a decrease in the detected antibody level(s) over time is indicative that the subject's ODBI or ODCI status is decreasing (improving) over time. In some embodiments, the detected antibody level(s) are levels of antibodies specific to the same deiminated variant(s). In some embodiments, the detected antibody level(s) are levels of antibodies specific to different deiminated variant(s) of the same protein(s). In some embodiments, the detected antibody level(s) are levels of antibodies specific to the same epitopes of the same deiminated variant(s). In some embodiments, the detected antibody level(s) are levels of antibodies specific to different epitopes of the same deiminated variant(s).

Also disclosed herein are methods of monitoring the progression of ODBI or ODCI in a subject, comprising: (a) applying a first biological sample taken from the subject to a first device comprising a plurality of deiminated peptides to detect antibody level(s) in the first biological sample, wherein at least one deiminated peptide comprises a deiminated portion of a deiminated variant of a protein listed in Table 1; (b) applying a second biological sample taken from the subject to a second device comprising a plurality of deiminated peptides to detect antibody level(s) in the second biological sample, wherein at least one deiminated peptide comprises a deiminated portion of a deiminated variant of a protein listed in Table 1; and (c) comparing the detected antibody level(s) in the first and second biological samples, wherein an increase in the detected antibody level(s) over time is indicative that the therapy is effective, and/or wherein a decrease in the detected antibody level(s) over time is indicative that the therapy is effective.

Also disclosed herein are methods of monitoring the progression of ODBI or ODCI in a subject, comprising:(a) applying a first biological sample taken from the subject to a first device comprising a plurality of deiminated peptides to detect antibody level(s) in the first biological sample, wherein at least one deiminated peptide comprises a deiminated portion of a deiminated variant of a protein listed in Table 2; (b) applying a second biological sample taken from the subject to a second device comprising a plurality of deiminated peptides to detect antibody level(s) in the second biological sample, wherein at least one deiminated peptide comprises a deiminated portion of a deiminated variant of a protein listed in Table 2; and (c) comparing the detected antibody level(s) in the first and second biological samples, wherein an increase in the detected antibody level(s) over time is indicative that the therapy is effective, and/or wherein a decrease in the detected antibody level(s) over time is indicative that the therapy is effective.

Also disclosed herein are methods of determining the efficacy of an ODBI-therapy or ODCI-therapy in a subject treated with the ODBI-therapy or ODCI-therapy, comprising: (a) analyzing at least two biological samples taken from the subject at different time points to detect level(s) of antibodies specific to a deiminated variant of one or more of the proteins listed in Table 1 or a fragment thereof; and (b) comparing the detected antibody level(s) in the biological samples taken at different time points, wherein an increase in the detected antibody level(s) over time is indicative that the therapy is not effective, and/or wherein a decrease in the detected antibody level(s) over time is indicative that the therapy is effective.

Also disclosed herein are methods of determining the efficacy of an ODBI-therapy or ODCI-therapy in a subject treated with the ODBI-therapy or ODCI-therapy, comprising: (a) analyzing at least two biological samples taken from the subject at different time points to detect level(s) of antibodies specific to a deiminated variant of one or more of the proteins listed in Table 2 or a fragment thereof, wherein the deiminated variant is deiminated at a site(s) listed in Table 2; and (b) comparing the detected antibody level(s) in the biological samples taken at different time points, wherein an increase in the detected antibody level(s) over time is indicative that the therapy is not effective, and/or wherein a decrease in the detected antibody level(s) over time is indicative that the therapy is effective.

Also disclosed herein are methods of determining the efficacy of an ODBI-therapy or ODCI-therapy in a subject treated with the ODBI-therapy or ODCI-therapy, comprising: (a) applying a first biological sample taken from the subject to a first device comprising a plurality of deiminated peptides to detect antibody level(s) in the first biological sample, wherein at least one deiminated peptide comprises a deiminated portion of a deiminated variant of a protein listed in Table 1; (b) applying a second biological sample taken from the subject to a second device comprising a plurality of deiminated peptides to detect antibody level(s) in the second biological sample, wherein at least one deiminated peptide comprises a deiminated portion of a deiminated variant of a protein listed in Table 1; and (c) comparing the detected antibody level(s) in the first and second biological samples, wherein an increase in the detected antibody level(s) over time is indicative that the therapy is effective, and/or wherein a decrease in the detected antibody level(s) over time is indicative that the therapy is effective.

Also disclosed herein are methods of determining the efficacy of an ODBI-therapy or ODCI-therapy in a subject treated with the ODBI-therapy or ODCI-therapy, comprising: (a) applying a first biological sample taken from the subject to a first device comprising a plurality of deiminated peptides to detect antibody level(s) in the first biological sample, wherein at least one deiminated peptide comprises a deiminated portion of a deiminated variant of a protein listed in Table 2; (b) applying a second biological sample taken from the subject to a second device comprising a plurality of deiminated peptides to detect antibody level(s) in the second biological sample, wherein at least one deiminated peptide comprises a deiminated portion of a deiminated variant of a protein listed in Table 2; and (c) comparing the detected antibody level(s) in the first and second biological samples, wherein an increase in the detected antibody level(s) over time is indicative that the therapy is effective, and/or wherein a decrease in the detected antibody level(s) over time is indicative that the therapy is effective.

In some embodiments of any of these methods, the method involves detecting antibodies that are specific to a deiminated variant of one or more of the proteins listed in Table 1 (or a fragment thereof). In some embodiments, the method involves detecting antibodies that are specific to a deiminated variant that is deiminated at the site(s) listed in Table 1. In some embodiments, the method additionally involves detecting antibodies to a deiminated variant of one or more of the proteins listed in Table 2 (or fragment thereof).

Additionally, or alternatively, in some embodiments of these methods, the method involves detecting antibodies that are specific to a deiminated variant of one or more of the proteins listed in Table 2 (or a fragment thereof) that is deiminated at the site(s) listed in Table 2.

In some embodiments of any of the foregoing embodiments, the method additionally involves detecting antibodies that are specific to a deiminated variant of one or more of the proteins listed in Table 3 (or a fragment thereof). In some embodiments of such embodiments, the method involves detecting antibodies that are specific to a deiminated variant that is deiminated at the site(s) listed in Table 3.

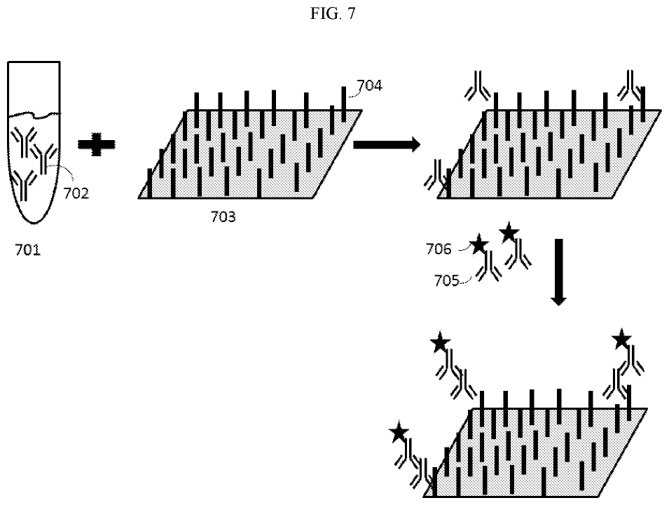

A schematic of an exemplary method for detecting antibodies specific to a deiminated variant of a protein listed in Tables 1-3 is depicted in FIG. 7. Deiminated peptides or respective native and/or deiminated proteins are printed at addressable locations to create a multiplex array. As shown in FIG. 7, a biological sample (701) comprising antibodies specific to deiminated variant(s) of one or more proteins described herein (referred to herein as autoantibodies) is applied to a protein/peptide array (703). The array comprises a plurality of different deiminated peptides or proteins (704). A wash buffer may be applied to the array to remove any non-adherent antibodies. A secondary detection antibody (705) is applied to the peptide array. Specifically, the secondary detection anti-human immunoglobulin (Ig) antibody is conjugated to a detection entity such as an enzyme, electron-generating reagent, fluorescent molecule or similar signal generating entity commonly used in microarray detection (706). A wash buffer may be applied to the array to remove any non-adherent secondary antibodies. The amount of anti-human Ig antibody bound to the peptide/protein array is detected, such as via the detectable label, and the identification and quantification of specific peptide/protein autoantibodies is determined by the location and intensity of the secondary antibody signal response.

### Treatment Methods

Disclosed herein are methods for treating an ODBI or ODCI in a subject in need thereof, wherein the subject may be identified as being in need of treatment by the methods disclosed above.

In some embodiments, such methods comprise administering to the subject a deiminase inhibitor. In some embodiments, such methods comprise administering to the subject an agent that inhibits an immune response to a deiminated protein as disclosed herein, such as an agent that inhibits an antibody specific to a deiminated variant of one or more of the deiminated proteins disclosed herein, such as an antibody that anti-idiotypic to an antibody specific to a deiminated variant of one or more of the deiminated proteins disclosed herein.

In some embodiments, the deiminase inhibitor inhibits or suppresses the activity of a protein arginine deiminase (PAD). In some embodiments, the PAD is selected from PAD 1, PAD 2, PAD 3, PAD 4, and PAD 6. Examples of deiminase inhibitors are disclosed in WO2007056389, WO2014019092, and WO2017027967, Bicker and Thompson, Biopolymers, 2013, 99(2):155-163, Nagar et al., Front Immunol. 2019 Feb 19;10:244, Jones et al., ACS Chemical Biology. 2011;7:160-165, Luo et al., Biochemistry. 2006;45:11727-11736, Causey et al., Journal of Medicinal Chemistry. 2011;54:6919-6935, Stone et al, Biochemistry. 2005;44:13744-13752, and Wang et al., J Biol Chem. 2012, 287(31):25941-53, which are incorporated by reference in their entirety. The deiminase inhibitor may be a reversible inhibitor, such as one or more reversible inhibitors disclosed in these references. The deiminase inhibitor may be an irreversible inhibitor, such as one or more irreversible inhibitors disclosed in these references. The deiminase inhibitor may be a second generation PAD inhibitor. The deiminase inhibitor may be selected from taxol, minocycline, streptomycin, o-F-amidine, o-Cl-amidine, Thr-Asp-F-amidine (TDFA), thioredoxin, and streptonigrin, or an analog or derivative of any thereof. In some embodiments, the deiminase inhibitor is a Cl-amidine analog, such as YW3-56.

Alternatively, or additionally, the method for treating an ODBI or ODCI in a subject in need thereof comprises administering one or more agents that inhibit one or more antibodies that bind to one or more deiminated proteins (or fragment thereof) listed in any of Tables 1, 2, and 3.

Thus, in some embodiments, the method for treating an ODBI or ODCI in a subject in need thereof comprises administering one or more agents that inhibit one or more antibodies that are specific to a deiminated variant of one or more of the proteins listed in Table 1 (or a fragment thereof). In some embodiments, the method for treating an ODBI or ODCI in a subject in need thereof comprises administering one or more agents that inhibit one or more antibodies that are specific to a deiminated variant that is deiminated at the site(s) listed in Table 1. In some embodiments, the method for treating an ODBI or ODCI in a subject in need thereof comprises administering one or more agents that inhibit one or more antibodies to a deiminated variant of one or more of the proteins listed in Table 2 (or fragments thereof). In some embodiments, the method for treating an ODBI or ODCI in a subject in need thereof comprises administering one or more agents that inhibit one or more antibodies that are specific to a deiminated variant of one or more of the proteins listed in Table 2 (or a fragment thereof) that is deiminated at the site(s) listed in Table 2. In some embodiments, the method for treating an ODBI or ODCI in a subject in need thereof comprises administering one or more agents that inhibit antibodies that are specific to a deiminated variant of one or more of the proteins listed in Table 3 (or a fragment thereof). In some embodiments, the method for treating an ODBI or ODCI in a subject in need thereof comprises administering one or more agents that inhibit antibodies that are specific to a deiminated variant that is deiminated at the site(s) listed in Table 3.

In some embodiments, a method for treating an ODBI or ODCI in a subject comprises: (a) analyzing a biological sample taken from the subject to detect antibodies specific to a deiminated variant of (i) one or more of the proteins listed in Table 1 (or a fragment thereof); (ii) one or more protein listed in Table 2 (or a fragment thereof); and/or (iii) one or more proteins listed in Table 3 (or a fragment thereof); (b) comparing the detected antibody level(s) to reference antibody level(s) of antibodies specific to a deiminated variant of the same protein(s) in a reference sample, wherein an increase in the detected antibody level(s) as compared to the reference antibody level(s) is indicative that the subject is suffering from ODBI or ODCI; and (c) administering to a subject indicated to be suffering from ODBI or ODCI a deiminase inhibitor, optionally wherein the deiminase inhibitor inhibits or suppresses the activity of a protein arginine deiminase (PAD), optionally wherein the PAD is selected from PAD 1, PAD 2, PAD 3, PAD 4, and PAD 6, optionally wherein the deiminase inhibitor is selected from taxol, minocycline, streptomycin, o-F-amidine, o-Cl-amidine, Thr-Asp-F-amidine (TDFA), thioredoxin, streptonigrin, and an analog or derivative thereof.

In some embodiments, a method for treating an ODBI or ODCI in a subject comprises: (a) applying a biological sample taken from the subject to a device comprising a plurality of deiminated peptides to detect antibody level(s) in the biological sample, wherein at least one deiminated peptide comprises a deiminated portion of a deiminated variant of: (i) a protein listed in Table 1; (ii) a protein listed in Table 2; and/or (iii) a protein listed in Table 3; (b) comparing the detected antibody level(s) to reference antibody level(s) of antibodies specific to a deiminated variant of the same protein(s) in a reference sample, wherein an increase in the detected antibody level(s) as compared to the reference antibody level(s) is indicative that the subject is suffering from ODBI or ODCI; and (c) administering to a subject indicated to be suffering from ODBI or ODCI a deiminase inhibitor, optionally wherein the deiminase inhibitor inhibits or suppresses the activity of a protein arginine deiminase (PAD), optionally wherein the PAD is selected from PAD 1, PAD 2, PAD 3, PAD 4, and PAD 6, optionally wherein the deiminase inhibitor is selected from taxol, minocycline, streptomycin, o-F-amidine, o-Cl-amidine, Thr-Asp-F-amidine (TDFA), thioredoxin, streptonigrin, and an analog or derivative thereof.

In some embodiments, a method for treating an ODBI or ODCI in a subject comprises: (a) analyzing a biological sample taken from the subject to detect antibodies specific to a deiminated variant of (i) one or more of the proteins listed in Table 1 (or a fragment thereof); (ii) one or more protein listed in Table 2 (or a fragment thereof); and/or (iii) one or more proteins listed in Table 3 (or a fragment thereof); (b) comparing the detected antibody level(s) to reference antibody level(s) of antibodies specific to a deiminated variant of the same protein(s) in a reference sample, wherein an increase in the detected antibody level(s) as compared to the reference antibody level(s) is indicative that the subject is suffering from ODBI or ODCI; and (c) administering to a subject indicated to be suffering from ODBI or ODCI an agent that inhibits an antibody specific to a deiminated variant of (i) one or more of the proteins listed in Table 1 (or a fragment thereof), optionally that inhibits an antibody specific to a deiminated variant that is deiminated at the site(s) listed in Table 1; (ii) one or more of the proteins listed in Table 2 (or a fragment thereof), optionally that inhibits an antibody specific to a deiminated variant that is deiminated at the site(s) listed in Table 2; and/or (iii) one or more of the proteins listed in Table 3 (or a fragment thereof), optionally that inhibits an antibody specific to a deiminated variant that is deiminated at the site(s) listed in Table 3, optionally wherein the agent is an anti-idiotypic antibody idiotypic to the antibody specific to the deiminated variant.

In some embodiments, a method for treating an ODBI or ODCI in a subject comprises: (a) applying a biological sample taken from the subject to a device comprising a plurality of deiminated peptides to detect antibody level(s) in the biological sample, wherein at least one deiminated peptide comprises a deiminated portion of a deiminated variant of: (i) a protein listed in Table 1; (ii) a protein listed in Table 2; and/or (iii) a protein listed in Table 3; (b) comparing the detected antibody level(s) to reference antibody level(s) of antibodies specific to a deiminated variant of the same protein(s) in a reference sample, wherein an increase in the detected antibody level(s) as compared to the reference antibody level(s) is indicative that the subject is suffering from ODBI or ODCI; and (c) administering to a subject indicated to be suffering from ODBI or ODCI an agent that inhibits an antibody specific to a deiminated variant of (i) one or more of the proteins listed in Table 1 (or a fragment thereof), optionally that inhibits an antibody specific to a deiminated variant that is deiminated at the site(s) listed in Table 1; (ii) one or more of the proteins listed in Table 2 (or a fragment thereof), optionally that inhibits an antibody specific to a deiminated variant that is deiminated at the site(s) listed in Table 2; and/or (iii) one or more of the proteins listed in Table 3 (or a fragment thereof), optionally that inhibits an antibody specific to a deiminated variant that is deiminated at the site(s) listed in Table 3, optionally wherein the agent is an anti-idiotypic antibody idiotypic to the antibody specific to the deiminated variant.

Further disclosed herein is a method for treating an ODBI or ODCI in a subject, comprising: administering a deiminase inhibitor to a subject determined to have elevated levels of antibodies specific to a deiminated variant of (i) one or more of the proteins listed in Table 1 (or a fragment thereof); (ii) one or more protein listed in Table 2 (or a fragment thereof); and/or (iii) one or more proteins listed in Table 3 (or a fragment thereof), as compared to reference antibody levels, optionally wherein the deiminase inhibitor inhibits or suppresses the activity of a protein arginine deiminase (PAD), optionally wherein the PAD is selected from PAD 1, PAD 2, PAD 3, PAD 4, and PAD 6, optionally wherein the deiminase inhibitor is selected from taxol, minocycline, streptomycin, o-F-amidine, o-Cl-amidine, Thr-Asp-F-amidine (TDFA), thioredoxin, streptonigrin, and an analog or derivative thereof.

Further disclosed herein is a method for treating an ODBI or ODCI in a subject, comprising: administering an agent to a subject determined to have elevated levels of antibodies specific to a deiminated variant of (i) one or more of the proteins listed in Table 1 (or a fragment thereof); (ii) one or more protein listed in Table 2 (or a fragment thereof); and/or (iii) one or more proteins listed in Table 3 (or a fragment thereof), as compared to reference antibody levels, wherein the agent inhibits an antibody specific to a deiminated variant of (i) one or more of the proteins listed in Table 1 (or a fragment thereof), optionally that inhibits an antibody specific to a deiminated variant that is deiminated at the site(s) listed in Table 1; (ii) one or more of the proteins listed in Table 2 (or a fragment thereof), optionally that inhibits an antibody specific to a deiminated variant that is deiminated at the site(s) listed in Table 2; and/or (iii) one or more of the proteins listed in Table 3 (or a fragment thereof), optionally that inhibits an antibody specific to a deiminated variant that is deiminated at the site(s) listed in Table 3, optionally wherein the agent is an anti-idiotypic antibody idiotypic to the antibody specific to the deiminated variant.

Any methods for treatment may further comprise determining the efficacy of the treatment, e.g., of the deiminase inhibitor(s) or agent(s) that inhibit one or more antibodies that are specific to a deiminated variant of one or more proteins disclosed herein, as disclosed above. Additionally or alternatively, any methods for treatment may further comprise monitoring the progression of ODBI or ODCI in the subject, as disclosed above.

### Devices For Detecting Antibodies To Deiminated Protein

Also disclosed herein are devices for detecting antibodies specific to the deiminated proteins described herein, such as antibodies specific to the deiminated peptides described herein, antibodies specific to an epitope comprising a deiminated arginine residue of the deiminated proteins described herein, and antibodies specific to any other deiminated variant of the deiminated proteins described herein. Generally, the devices may comprise one or more deiminated peptides based on the deiminated proteins described herein (or a fragment thereof). The devices disclosed herein may be used to detect antibodies specific to the deiminated proteins described herein, or to diagnose or monitor ODBI or ODCI in a subject, as discussed above.

In some embodiments, the device comprises a deiminated peptide that comprises at least a deiminated portion of a deiminated variant of a protein listed in Table 1, or a deiminated fragment thereof. In some embodiments, the device comprises one or more deiminated peptides that comprise one or more deiminated portions of one or more deiminated variants of one or more proteins listed in Table 1, or a deiminated fragment thereof. In some embodiments, the deiminated variant is deiminated at a site listed in Table 1. In some embodiments, the deiminated portion of the deiminated variant of the protein listed in Table 1 comprises at least 10%, 20%, 30%, 40%, 50%, 60%, 70%, 80%, 90%, or 100% of the amino acid sequence of the deiminated protein listed in Table 1. In some embodiments, the device additionally comprises one or more deiminated peptides comprising one or more deiminated portions of one or more deiminated variants of one or more proteins listed in Table 2, or a deiminated fragment thereof. In some embodiments, the deiminated variant is deiminated at a site listed in Table 2.

Additionally, or alternatively, in some embodiments, the device comprises a deiminated peptide that comprises at least a deiminated portion of a deiminated variant of one or more of the proteins listed in Table 2 that is deiminated at one or more of the site(s) listed in Table 2 (or a fragment thereof comprising a listed deiminated site). In some embodiments, the deiminated portion of the deiminated variant of the protein listed in Table 2 comprises at least 3 amino acids and up to 10%, 20%, 30%, 40%, 50%, 60%, 70%, 80%, 90%, or 100% of the amino acid sequence of the deiminated protein listed in Table 2.

In some embodiments of any of the foregoing embodiments, the device additionally comprises a deiminated peptide that comprises at least a deiminated portion of a deiminated variant of one or more of the proteins listed in Table 3 (or a fragment thereof comprising a deiminated site). In some embodiments of such embodiments, the device comprises a deiminated peptide that comprises at least a deiminated portion of a deiminated variant of one or more proteins listed in Table 3 that is deiminated at one or more of the site(s) listed in Table 3 (or a fragment thereof comprising a listed deiminated site). In some embodiments, the deiminated portion of the deiminated variant of the protein listed in Table 3 comprises at least 3 amino acids and up to 10%, 20%, 30%, 40%, 50%, 60%, 70%, 80%, 90%, or 100% of the amino acid sequence of the deiminated protein listed in Table 3.

### Deiminated Proteins and Peptides

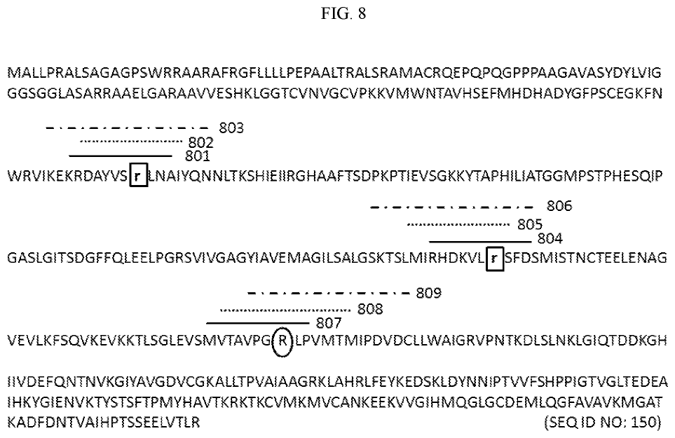

Tables 1-3 set forth proteins that are deiminated in response to oxygen deprivation in human brains, and identify specific regions thereof that are deiminated at specific deimination sites (specific arginine residues). Deiminated peptides based on these proteins may be synthesized and used in the methods and devices described herein. A deiminated peptide may be of any suitable length for detecting (e.g., being bound by) antibodies to the deiminated protein, such as from 3-50 amino acid residues, and may comprise any suitable number of amino acid residues flanking the deiminated arginine residue(s), such as from 1-49 amino acid flanking residues on either side. In some embodiments, the deiminated peptides comprise a secondary or tertiary structure that is suitable for detecting (e.g., being bound by) antibodies to the deiminated proteins. In such case, the deiminated peptides may comprise greater than 50 amino acid residues of the deiminated proteins. In some embodiments, the deiminated peptide comprises 55-100, 100-200, 200-500, 500-1000, 1000-1500, 1500-2000 or more amino acid residues of the deiminated protein, and may comprise any suitable number of amino acid residues flanking the deiminated arginine residue(s), such as from 1-2000 amino acid flanking residues on either side. In some embodiments, the deiminated peptide comprises 3 amino acids and up to 10%, 20%, 30%, 40%, 45%, 50%, 55%, 60%, 65%, 70%, 75%, 80%, 85%, 90%, 95% or 100% of the amino acid sequence of a deiminated protein listed in Tables 1-3. Example 3 and FIG. 8 illustrate the design of deiminated peptides as described herein.

For illustration, FIG. 8 shows the protein sequence for glutathione reductase, mitochondrial isoform 1 precursor (accession number: NP_000628.2) with the two identified deiminated arginine residues shown in boxed, lowercase “r”. The two identified deiminated arginine residues correspond to R153 and R268 of the published sequence for accession number: NP_000628.2. As shown in FIG. 8, a deiminated peptide (e.g., peptides 801-806) may comprise an identified deiminated arginine residue and flanking amino acid residues. Alternatively, as also shown in FIG. 8, a deiminated peptide (e.g., peptides 807-809) may be based on an arginine residue that has not been confirmed as a deiminated arginine residue, but is a possible deiminated arginine residue (see circled, uppercase “R”). In such embodiments, the deiminated peptide (e.g., peptides 807-809) may comprise the possible deiminated arginine residue and flanking amino acid residues.

In some embodiments, a deiminated peptide comprises at least a deiminated portion of a deiminated variant of a protein listed in any one of Tables 1-3. In specific embodiments, a deiminated peptide comprises at least a deiminated portion of a deiminated variant of a protein listed in Table 1. In specific embodiments, a deiminated peptide comprises at least a deiminated portion of a deiminated variant of a protein listed in Table 2. In specific embodiments, a deiminated peptide comprises at least a deiminated portion of a deiminated variant of a protein listed in Table 3.

In some embodiments, a deiminated peptide comprises at least a deiminated portion of a deiminated protein listed in any one of Tables 1-3 that is deiminated at least one of the site(s) set forth in Tables 1-3, respectively. In specific embodiments, a deiminated peptide comprises at least a deiminated portion of a deiminated variant of a protein listed in Table 1, deiminated at at least one of the site(s) set forth in Table 1. In specific embodiments, a deiminated peptide comprises at least a deiminated portion of a deiminated variant of a protein listed in Table 2, deiminated at at least one of the site(s) set forth in Table 2. In specific embodiments, a deiminated peptide comprises at least a deiminated portion of a deiminated variant of a protein listed in Table 3, deiminated at at least one of the site(s) set forth in Table 3. In further specific embodiments of these embodiments, the deiminated peptide further comprises one or more of the flanking residues on each side of the deiminated arginine residue(s) set forth in Tables 1-3, respectively. In further specific embodiments, a deiminated peptide comprises or consists of a deiminated peptide listed in any one of Tables 1-3, or a fragment thereof comprising a deiminated site. In some embodiments, a deiminated peptide comprises or consists of a deiminated peptide listed in Table 1, or a fragment thereof comprising a deiminated site. In some embodiments, a deiminated peptide comprises or consists of a deiminated peptide listed in Table 2, or a fragment thereof comprising a deiminated site. In some embodiments, a deiminated peptide comprises or consists of a deiminated peptide listed in Table 3, or a fragment thereof comprising a deiminated site.

In some embodiments, the methods or devices indirectly detect a plurality of deiminated proteins described herein. For example, the methods or devices may detect a plurality of different antibodies, each being specific to a different deiminated variant of a protein described herein, and the devices may comprise a plurality of different deiminated peptides based on different deiminated variants of the proteins described herein, such as at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more. In some embodiments, the devices and methods detect 2 or more different antibodies. In some embodiments, the devices and methods detect 5 or more different antibodies. In some embodiments, the method comprises detecting 10 or more different antibodies. In some embodiments, the devices and methods 15 or more different antibodies. In some embodiments, the devices and methods detect 20 or more different antibodies. In some embodiments, the devices and methods detect 30 or more different antibodies. In some embodiments, the devices and methods detect 40 or more different antibodies. In some embodiments, the devices and methods detect 50 or more different antibodies. In some embodiments, the devices and methods detect 60 or more different antibodies. In some embodiments, the devices and methods detect 70 or more different antibodies. In some embodiments, the devices and methods detect 80 or more different antibodies. In some embodiments, the devices and methods detect 90 or more different antibodies. As used here “different antibodies” denotes antibodies specific to different deiminated peptides or proteins. The different deiminated variants may therefore be deiminated variants of different proteins, or may be deiminated variants of the same protein that are deiminated at different sites. The methods or devices may detect a plurality of different antibodies specific to deiminated variants of the proteins set forth in Table 1-3, in accordance with the descriptions above.

Thus, in some embodiments, a device as described herein comprises one or more deiminated peptides as described herein. The deiminated peptides may be selected from those set forth in Table 1-3 in accordance with the descriptions above. A device may comprise a plurality of different deiminated peptides in accordance with the description above.

Likewise, in some embodiments, a method as described herein comprises detecting antibodies in a biological sample by contacting the biological sample with one or more deiminated peptides as described herein to thereby detect antibodies to one or more deiminated variant(s) of the proteins described herein. The detected antibodies may be selected from those set forth in Table 1-3 in accordance with the descriptions above. A plurality of different antibodies may be detected in accordance with the description above.

In some embodiments of the devices and methods, the deiminated peptides are attached to a solid support, such as a slide, plate, well, array, chip, or particle. In some embodiments, the slide is a microarray slide. In some embodiments, the solid support is a microwell. In some embodiments, the solid support is a multi-array plate. In some embodiments, the multi-array plate comprises 96 or more wells. In some embodiments, the multi-array plate comprises 384 or more wells. In some embodiments, the solid support is a particle, such as a spherical particle. In some embodiments, the spherical particle is a bead. In some embodiments, the bead is a magnetic bead, streptavidin bead, agarose bead, silica bead, gold bead, glass bead, or amino bead. In some embodiments, the magnetic bead is a magsilica bead, agarose matrix bead, or carboxyl magnetic bead. In some embodiments, the agarose bead is a nickel agarose bead. In some embodiments, the silica bead is a hydroxyl silicon bead.

In some embodiments, the deiminated peptides are covalently attached to the solid support. In some embodiments, the deiminated peptides are non-covalently attached to the solid support. In some embodiments, the deiminated peptides are directly attached to the solid support. In some embodiments, the deiminated peptides are indirectly attached to the solid support. For example, the deiminated peptides may be indirectly attached to the solid support via an affinity linker or adapter.

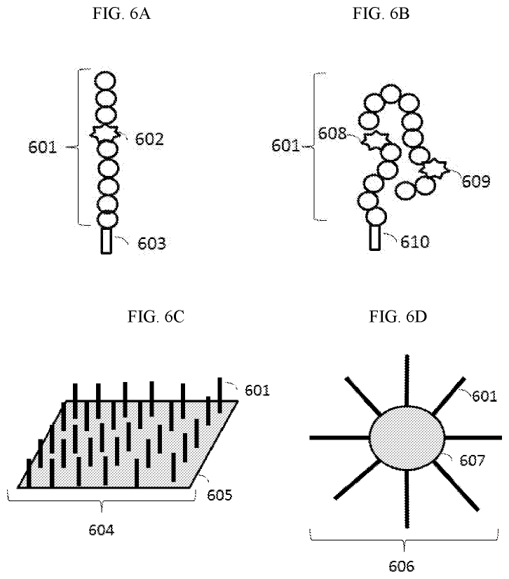

In some embodiments, deiminated peptides and antibodies bind to form an antibody-deiminated peptide complex that can be detected using the devices and methods disclosed herein. In some embodiments, the deiminated peptides are conjugated to a detection moiety such that formation of the antibody-deiminated peptide complex results in detectable signal, such as a decrease in the intensity of the detection moiety. In some embodiments, the antibody-deiminated peptide complex is detected by the difference in weight and/or pressure as compared to the uncomplexed deiminated peptide. FIG. 6 shows schematics of a deiminated peptide and deiminated peptides attached to a solid support. As shown in FIG. 6A, a deiminated peptide (601) comprises a plurality of amino acids, wherein at least one of the amino acids is a deiminated arginine residue (602). The deiminated peptide (601) may be attached to linker or adaptor (603), which can be used to indirectly attach the deiminated peptide to a solid support. As shown in FIG. 6B, a deiminated peptide (601) comprises a secondary or tertiary structure. The deiminated peptide (601) comprises a plurality of amino acids, wherein one or more of the amino acids is a deiminated arginine residue (608, 609). The deiminated peptide comprising a secondary or tertiary structure (601) may be attached to a linker or adaptor (603), which can be used to indirectly attach the deiminated peptide to a solid support. As shown in FIG. 6C, the deiminated peptide (601) may be attached to a solid support (605, e.g., a microscope slide) to produce a peptide array (604). Alternatively, as shown in FIG. 6D, the deiminated peptide (601) may be attached to a solid support (607, e.g., spherical particle) to produce a peptide-conjugated bead (606).

Deiminated peptides may be synthesized by methods known in the art. For instance, deiminated peptides may be synthesized by liquid-phase peptide synthesis or solid-phase peptide synthesis using citrulline protected by fluorenylmethyloxycarbonyl (Fmoc) in place of Fmoc protected arginine. Methods of liquid-phase peptide synthesis are described, for example, in Fischer P M and Zheleva D I, “Liquid-phase peptide synthesis on polyethylene glycol (PEG) supports using strategies based on the 9-fluorenylmethoxycarbonyl amino protecting group: application of PEGylated peptides in biochemical assays,” J. Pept. Sci., 2002, 8(9):529-42; Takahashi D, et al., “AJIPHASE®: A Highly Efficient Synthetic Method for One-Pot Peptide Elongation in the Solution Phase by an Fmoc Strategy,” Angew Chem. Int. Ed. Engl., 2017, 56(27):7803-7807, and Okada Y, et al., “Tag-assisted liquid-phase peptide synthesis using hydrophobic benzyl alcohols as supports,” J. Org. Chem., 2013, 78(2):320-7. Methods of solid-phase peptide synthesis are described, for example, in Merrifield R M, “Solid Phase Peptide Synthesis. I. The Synthesis of a Tetrapeptide,” J. Am. Chem. Soc., 1963, (14): p.2149-2154; Moss J A, “Guide for resin and linker selection in solid-phase peptide synthesis,” Curr. Protoc. Protein. Sci., 2005 Jun; Chapter 18:Unit 18.7.ps1807s40, and Wang, S. S., “p-alkoxybenzyl alcohol resin and p-alkoxybenzyloxycarbonylhydrazide resin for solid phase synthesis of protected peptide fragments,” J. Am. Chem. Soc., 1973, (4): p.1328-33.

Deiminated peptides may be synthesized directly onto an array. For example, a peptide array may be generated based on the principle of in situ photolithographic synthesis in a Roche-Nimblegen Maskless Array Synthesizer (MAS), as described in Shin DS, et al., “Automated maskless photolithography system for peptide microarray synthesis on a chip,” J. Comb. Chem. 2010, 12 (4), 463-71, and Zandian A, et al., “Whole-proteome peptide microarrays for profiling autoantibody repertoires within multiple sclerosis and narcolepsy,” J. Proteome Res., 2017, 16(3):1300-1314.

As discussed above, the devices and methods disclosed herein detect antibodies that are specific to a deiminated variant of one or more of the proteins listed in Tables 1-3 (or a fragment thereof). In some embodiments, the antibodies detected by the devices and methods disclosed herein are immunoglobulin gamma (IgG) antibodies. In some embodiments, the antibodies are immunoglobulin mu (IgM) antibodies. In some embodiments, the antibodies are human antibodies.

In some embodiments, the antibodies detected by the devices and methods disclosed herein are specific to a deiminated arginine residue of any of the proteins described herein. Alternatively, or additionally, the antibodies detected by the devices and methods disclosed herein may be specific to one or more amino acid residues adjacent to or surrounding a deiminated arginine residue of any of the proteins described herein. The deiminated arginine residue may correspond to a deiminated arginine residue identified in Table 1. In some embodiments, the deiminated arginine residue corresponds to a deiminated arginine residue identified in Table 2. In other embodiments, the deiminated arginine residue corresponds to a deiminated arginine residue identified in Table 3. Alternatively, or additionally, the deiminated arginine residue may correspond to any arginine residue of any of the proteins listed in Table 1. In some embodiments, the deiminated arginine residue corresponds to any arginine residue of any of the proteins listed in Table 2. In other embodiments, the deiminated arginine residue corresponds to any arginine residue of any of the proteins listed in Table 3.

In some embodiments, the methods involving using one or more secondary antibodies to detect antibodies to deiminated proteins described herein, such as to detect antibodies bound to deiminated peptides on a device as described herein. In some embodiments, the secondary antibodies are specific to the human autoantibodies. In some embodiments, the secondary antibodies are anti-human immunoglobulin G (anti-IgG) antibodies. In some embodiments, the secondary antibodies are anti-human immunoglobulin M (anti-IgM) antibodies). In some embodiments, the secondary antibodies are specific to the heavy chain of the antibodies.

In some embodiments, the secondary antibodies are conjugated to a detection moiety. In some embodiments, the detection moiety is selected from, an enzyme, electron generating molecule, electrochemiluminescent tag, fluorescent tag, luminescent tag. In some embodiments, the enzyme label is selected from horseradish peroxidase (HRP), alkaline phosphatase (AP), glucose oxidase and β-galactosidase. In some embodiments, the electron generating molecule and thus, electochemiluminescent tag is ruthenium.

In some embodiments, the antibodies or antibody-deiminated peptide complexes are detected by immunodetection, chemiluminescence, fluorescence, bioluminescence, electrochemiluniscence, or chemifluorescence detection. In some embodiments, the antibodies are detected by electrochemiluminescence.

### Biological Sample

In some embodiments, the biological sample is selected from blood, cerebrospinal fluid (CSF) or saliva. In some embodiments, the biological sample is blood. In some embodiments, the blood is peripheral blood, serum, or plasma.

In some embodiments, the biological sample is taken from the subject prior to the subject receiving an ODBI-therapy or ODCI-therapy. In other embodiments, the sample is taken from the subject after the subject has received an ODBI-therapy or ODCI-therapy.

In some embodiments, 1, 2, 3, 4, 5, 6, 7, 8, 9, or 10 or more biological samples are taken from the subject. In some embodiments, two or more biological samples are taken from the subject. In some embodiments, three or more biological samples are taken from the subject.

In some embodiments, the biological samples are taken from the subject once every week, 2 weeks, 3 weeks, 4 weeks, 5 weeks, 6 weeks, 2 months, 3 months, 4 months, 5 months or 6 months or more. In some embodiments, the biological samples are taken from the subject for a period of at least 2 weeks, 3 weeks, 1 month, 2 months, 3 months, 4 months, 5 months, or 6 months or more.

### Subject

In some embodiments, the subject is suspected of or at risk of having ODBI or ODCI. In some embodiments, the ODCI is traumatic brain injury (TBI). In some embodiments, the TBI is selected from mild TBI (mTBI) or severe TBI (sTBI), closed head injury (CHI), and blast-induced traumatic brain injury (bTBI). In some embodiments, the TBI is selected from a concussion, contusion, coup-contrecoup injury, diffuse axonal injury, and penetration injury. In accordance with specific embodiments, the TBI is blast-induced traumatic brain injury (bTBI).

In some embodiments, the subject is a human. In some embodiments, the subject is an adult. In some embodiments, the subject is a juvenile. In some embodiments, the subject is a child. In some embodiments, the subject is at least 18 years old. In some embodiments, the subject is less than 18 years old. In some embodiments, the subject is a soldier or other military personnel, law enforcement officer, or athlete. In some embodiments, the subject is employed in a physically dangerous job that may put the subject at risk of suffering from ODBI or ODCI.

In some embodiments, the subject is treated with an ODBI-therapy and/or an ODCI-therapy. In some embodiments, the ODBI-therapy and/or the ODCI-therapy comprises one or more anti-immune drugs and/or anti-idiotypic antibodies.

### EXAMPLES

### Example 1. Identification of Traumatic Brain Injury Markers in Animal Models

### Materials and Methods

### Animals

Studies were conducted in adult male Yucatan miniature and Yorkshire swine (Sinclair Bio Resources, LLC., and Archer Farms, Darlington, Md., respectively) weighing 40-50 kg, N=4/group). Animals were cared for and treated in accordance with guidelines approved by the US Department of Agriculture and the Medical Research and Material Command of the US Army.

Anesthetized pigs in the injured group were positioned in sternal recumbency and equipped with specially made Kevlar and lead body armor as well as head and face protection. Animals were then transferred to a blast tube, simulating free-field blast, where they received a single moderate blast overpressure exposure (40-52 psi, average=46 psi). Details pertaining to anesthesia, pre- and post-procedural treatments, and blast structure were as described earlier [5, 35-37]. Immediately after blast exposure, animals were removed from the blast structure and returned to the adjacent procedure facility for recovery. The endotracheal tube was maintained until animals exhibited normal pharyngeal function via cough reflex. Physiological parameters were electronically recorded by the monitoring system until animals were fully recovered from anesthesia and returned to their holding cages. In the days that followed, animals were assessed for pain or distress and monitored for general health as well as cranial nerve, neurologic and respiratory functions.

All animals were euthanized 2 weeks after exposure and whole brains were collected and rinsed in physiologic saline. Coronal sections were prepared (−0.75 inch thick), snap frozen on dry ice, and stored at −80° C. until used.

### Sample Preparation

Brain sections of frontal cortex were thawed and further dissected to produce wedges of tissue that contained an equivalent representation of all layers of cerebral cortex frontal lobe. Tissue samples were homogenized in 5 volumes/tissue weight of 0.1 M Tris buffer (pH 7.4) containing 1×complete protease inhibitors using a Polytron (setting 6; 3×15 second pulses, with chilling in between) (Roche; Basel, Switzerland) followed by 3 freeze-thaw cycles and centrifugation (20,000×g, 15 min, 4° C.). The resulting supernatants were removed and stored at −80° C. until used.

### Liquid-Phase Isoelectric Focusing

Liquid-phase isoelectric focusing (LP-IEF) of brain supernatants was carried out as previously described [31]. Briefly, treatment group pools (naïve and blast, N=4/group) underwent concentration and buffer exchange to water/1×protease inhibitors by Vivaspin (10 kDa molecular weight cut off (MWCO); General Electric, Fairfield, Conn.), removing TRIS which interferes with LP-IEF. Samples were then diluted in 1.1×IEF running solution (7.7 M urea, 2.2 M thiourea, and 4.4% CHAPS) and 1×complete protease inhibitor (1 part sample/9 parts IEF buffer). Samples were further adjusted for IEF fractionation by combining 900 μL pooled sample with ampholytes (150 μL, pH 3-10; Novex, Thermo Fisher, ZM0021; Waltham, Mass.), dithiolthreitol (DTT; 25 μL, 4 M) and bromphenol blue (20 μL, 10 mg/ml ). IEF fractionation was performed under the following conditions: (1) 100V, 2 mA, 2 W (20 min); (2) 200V, 2 mA, 2 W (80 min), (3) 400V, 2 mA, 2 W (80 min), (4) 600V, 2mA, 2W (80 min) using the ZOOM IEF Fractionator (Thermo Fisher; Waltham, Mass.). The resulting fractionation produced samples corresponding to the predicted IEF pH ranges for the fractionator (pH 3.0-4.6, pH 4.6-5.4, pH 5.4-6.2, pH 6.2-7.0, and pH 7.0-9.1) as judged by pH testing using pH strips. 1-dimensional gel electrophoresis and Commassie staining (see below) were used to confirm equivalent fraction profiles for the naïve and blast samples and to verify equivalent protein concentrations for naïve and blast samples of the same pH range.

### 1-Dimensional Gel Electrophoresis

IEF fractions were further resolved by molecular weight fractionation using conventional sodium dodecyl sulfate polyacrylamide gel electrophoresis (SDS-PAGE). This two-step reduction in the complexity of the proteome by IEF and SDS-PAGE was important for visualization of deiminated proteins by western blot analysis. Briefly, IEF samples were diluted in 4× reducing loading buffer (10% LDS, 10% glycerol, 0.4 M DTT, 250 mM Tris buffer, 20 μL bromphenol blue (10 mg/mL), pH 8.4), heated at 70° C. (10 min), and then fractionated in NuPage 4-12% Bis-Tris gels (Novex, Thermo Fisher; Waltham, Mass.), using 1×MES (2-[N-morpholino] ethanesulfonic acid) running buffer (9.76 gm/L MES, 60.6 gm/L Tris Base, 0.3 gm/L disodium ethylenediaminetetraacetic acid (EDTA), and 1 gm/L SDS, pH 1 8). Proteins were transferred to nitrocellulose using an iBlot transfer system (Thermo Fisher, Waltham, Mass.).

### Immunoblotting

Protein Deimination: Nitrocellulose membranes were blocked with 5% non-fat dry milk/Tris buffered saline/Tween 20 (TBS-T) (25 mM Tris Base, 0.115 M NaCl, 25 mM KCl, 0.1% Tween20, pH 7.5) for 2 h at room temperature and then incubated overnight at 4° C. with mouse monoclonal anti-protein citrulline primary antibody 6B3 [31, 38] (stock=1.79 mg/mL) diluted (1:500) in 5% non-fat dry milk/TBS-T. Membranes were then washed in TBS-T (3 times over 60 min), incubated with secondary antibody (HRP conjugated goat anti-mouse IgG (H+L), 31430, 1:2500 in TBS-T; ThermoFisher, Waltham, Mass.) at room temperature for 2 h. Membranes were then washed in TBS-T (3 times over 60 min) and then visualized with enhanced chemiluminscence (ECL) (Novex ECL HRP Chemiluminescent Substrate Reagent Device; WP20005, Invitrogen,

Thermo Fischer; Waltham, Mass.) using the ChemiDoc Touch imaging system (Bio-Rad Laboratories, Hercules, Calif.). The specificity of the 6B3 mAb for detecting deiminated proteins was verified as described previously [31]. Images collected were analyzed using Image Lab software (v5.2.1, Bio-Rad Laboratories; Hercules, Calif.). Anti-peptidyl-citrulline, clone F95 antibody was obtained commercially from Millipore (ab# MABN328, Darmstadt, Germany) and used similarly.

Tissue IgG: Nitrocellulose membranes were blocked with 5% non-fat dry milk in TBS-T (2 h, room temperature) and then incubated with goat anti-porcine IgG (H+L), HRP conjugated antibody (1:2500 in TBS-T, EMD Millipore; Billerica, Mass., AP166P) at room temperature for 2 h. Membranes were then washed in TBS-T (3 times over 60 min) and then visualized and analyzed as described for Protein Deimination above. Quantitation of the ECL Western blot signals was based upon standardization to protein load for each sample, as determined by the signal density for equivalent samples visualized on Coomassie-stained gels. ImageJ (Mac Version 1.50i, National Institutes of Health) was used to determine both ECL and Coomassie data; signal densities of immunoreactive ECL features (heavy and light chain bands) were summed and adjusted for protein load based upon a percent difference from a reference protein load (highest protein load of control samples=100%).

### Protein Identification and Mapping of Deimination Sites

Immunoreactive signals of interest were mapped to corresponding banding patterns of the Coomassie-stained gels. The bands were excised and analyzed by peptide mass finger printing and tandem mass spectrometry (MS-MS) using the proteomic services of the W. M. Keck Foundation Biotechnology Resource Laboratory (New Haven Conn., USA). Briefly, gel bands were cut into smaller pieces, digested with trypsin, peptides extracted and desalted, and analyzed by liquid chromatography (LC) MS-MS using an Orbitrap Fusion Tribrid mass spectrometer (Thermo Fischer; Waltham, Mass.). Both MS and MS/MS scans were acquired in an Orbitrap analyzer. MS scans were of m/z range 350-1550, resolution of 120,000, AGC (automatic gain control) target of 2e5, and maximum injection time of 60 ms, while MS/MS scans were with fixed first mass of m/z 120, resolution of 60,000, AGC target of 5e4, and maximum injection time of 110 ms. Precursor ions were fragmented by high energy collision-induced dissociation collision energy (%) set to 28.

The raw files were processed using Proteome Discoverer v2.1 (Thermo Fischer, Waltham, Mass.) software. The files were searched with Sequest HT algorithm against pig UniProt database (downloaded June 2016). The fragment ion mass tolerance of 0.02 Da, parent ion tolerance of 10 ppm, digestion enzyme trypsin were specified in the Sequest analysis parameters. Oxidation of methionine, deamidation of asparagine and glutamine, deimination (deimination) of arginine, and propionamide of cysteine were specified in Sequest as variable modifications.

Scaffold software (v4.6.1, Proteome Software) was used to validate peptide and protein identifications. Initial protein analysis was performed with a protein threshold of 99%, a minimum of 3 peptides, and a peptide threshold of 95%. The inclusion criteria for peptides with a deiminated arginine, were an Xcorr of 2 or more and a deltaCn of >0.4. Peptides meeting these criteria were then assessed for a 43 Da neutral loss assessing for the loss of isocyanic acid (HNCO) through spectrometric analysis described by Hao et al [37]. All spectra were visually reviewed to insure quality and clear presence of the 43 Da neutral loss signature for deimination.

The expected mass of the neutral loss of isocyanic acid was determined by subtracting the product of a 43 Da loss divided by the peptide charge from the observed mass of the peptide sequence [37]. Peaks corresponding to the expected neutral loss were identified in the spectrum data and included if an observed peak was less than 2 Da from the expected peak. The only exception to this was GABA transaminase (4-aminobutyrate aminotransferase) that had peaks identified at approximately 3 Da from the expected neutral loss mass. This tentative identification was included here based on the high quality of the spectrum, Xcorr, and deltaCn.

### Statistical Analysis

Quantitation of the IgG ECL Western blot signals was based on standardization to protein load for each sample, as determined by the signal density for equivalent samples visualized on Coomassie-stained gels. ImageJ (Mac Version 1.50i, National Institutes of Health) was used to determine both ECL and Coomassie data; signal densities of immunoreactive ECL features (heavy and light chain bands) were summed and adjusted for protein load based on a percent difference from a reference protein load (highest protein load of control samples=100%). Analysis of the combined relative signal intensity of the IgG naive and blast results was performed using Prism 7 for Mac OS X (v7.0a, GraphPad Software, Inc). An unpaired t-test was performed with the standard variance assumed to be equal in the population. Statistical significance was determined using the Holm-Sidak method with an alpha of 0.05.

### Results

FIGS. 2A-B show the effects of blast exposure on the status of protein deimination in the porcine cerebral cortex. Brains were collected 2 weeks post-blast exposure and cerebral cortex homogenates were prepared from each subject. Treatment group pools of sham and blast samples, representing 4 animals each, underwent two-dimensional fractionation involving liquid phase isoelectric focusing (LP-IEF) followed by molecular weight fractionation using 1-dimensional sodium dodecyl sulfate polyacrylamide gel electrophoresis (SDS-PAGE). These steps for reducing the complexity of the proteome were necessary to clearly reveal western blot signals in the analyses of protein deimination. FIG. 2A shows the Coomassie-stained protein profiles for the control and blast groups over the four LP-IEF pH fractions. The data show that LP-IEF yielded pH fractions with distinct protein profiles, indicating the effectiveness of the IEF procedure to separate a complex proteome into sub-fractions having reduced protein complexity. It was also observed that within a given pH fraction, there was no apparent difference in the Coomassie banding profile between the control and blast samples with the exception of feature 11. This feature, which had increased Coomassie staining in the blast-exposed fraction, was determined to contain IgG by both mass spectrometric and western blot analyses.

The effects of blast exposure on the profile of protein deimination, using 6B3 antibody western blotting, is shown in FIG. 2B. These data indicate that there is a basal level of protein deimination in the control condition that involves a small subset of the proteins making up the entire brain proteome. Further, blast exposure dramatically affected the deimination status of several, but not all of these features. In most cases blast exposure resulted in a pronounced increase in the observed deimination signal (features 2-11), in some cases increasing from virtually no signal in the control condition (feature 9). There was also evidence for blast exposure in reducing the degree of protein deimination within a protein band, as can be most clearly seen for feature 1. Preliminary findings with an alternative anti-protein citrulline antibody, F95, identified some features that were not revealed by antibody 6B3, and vice versa (not shown), suggesting that the amino acid context of a deimination site contributes to its antibody recognition. Additionally, it was observed that deimination signals observed by western blot were reduced upon repeated freeze-thaw cycles of the samples, indicating the importance of preparing sample aliquots for storage and repeat analyses.

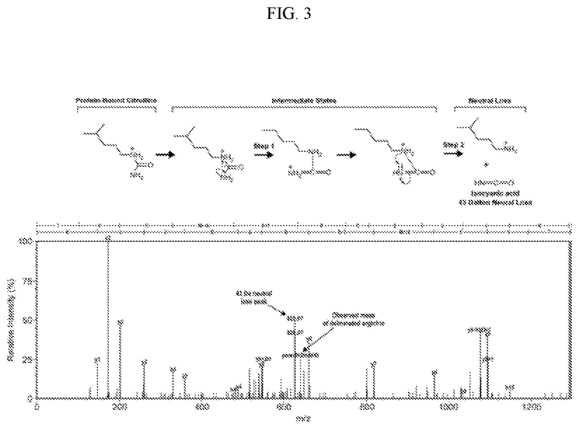

Immunoreactive features identified by 6B3 Western blotting (FIG. 2B; features 1-11) were mapped to corresponding protein bands in a replicate Coomassie-stained protein gel (FIG. 2A, features 1-11). These bands were collected and analyzed proteomically to identify the proteins present and to map their respective deimination sites. Site-specific deimination was confirmed by the demonstration of neutral loss of 43 Da representing the signature deimination fragment isocyanic acid [37] (FIG. 3) (see Materials and Methods for details). Table 4 presents a list of the 6 proteins definitively identified and their respective deimination sites. The findings include deiminated GFAP and vimentin, both of which are recognized as autoantigens in neuropathology [39] and rheumatoid arthritis [40], respectively.

TABLE 4
Mapping deimination sites in brain proteins of swine to repeated blast exposure.
Table 4 discloses SEQ ID NOS 143, 152 and 145-149, respectively, in order of appearance.

| | | | | Expected | |
|---|---|---|---|---|---|
| | | | | Mass | |
| | | | | with | Mass of |
| | | Observed | Charge | Neutral | Peak |
| Protein | Peptide Sequence | Mass | State | Loss | Detected |
| GABA | LVQQPQNVSTFINRPALGILPPENFVEK | 1050.57 | 3 | 1036.24 | 1033.26 |
| Transaminase | | | | | |
| Aconitase | LNRPLTLSEK | 391.23 | 3 | 376.89 | 376.67 |
| Hydratase | | | | | |
| Glial Fibrillary | ITIPVQTFSNLQIRETSLDTK | 802.43 | 3 | 766.10 | 789.75 |
| Acidic Protein | TVEMRDGEVIK | 647.32 | 2 | 525.82 | 825.82 |
| Glutathione S- | AFLASPEHVNRPINGNGK | 481.25 | 4 | 470.50 | 459.23 |
| transferase | | | | | |
| Histone H4 | SGLIYEETRGVLKVFLENVIRDAVTYTEHAK | 733.80 | 5 | 725.20 | 726.32 |
| Vimentin | TVETRDGQVINETSQHHDDLE | 808.70 | 3 | 794.37 | 794.37 |

R = deimination site

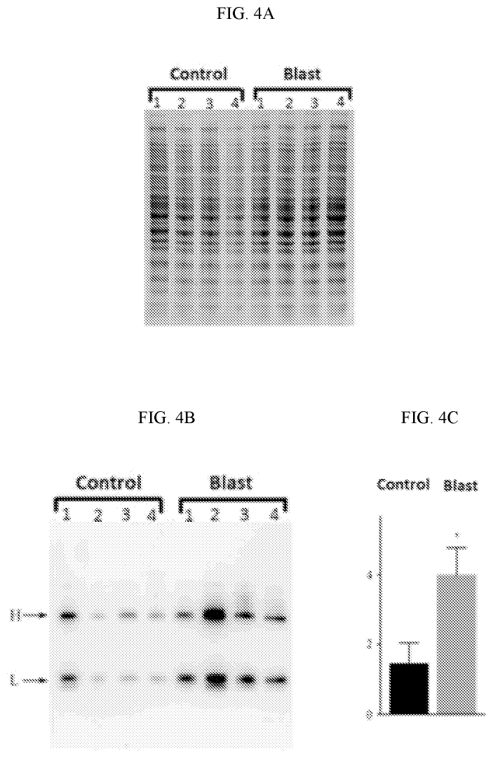

Western blotting for protein deimination employed an anti-mouse IgG detection antibody that was subsequently shown to cross-react with porcine IgG. This reagent revealed an intense signal in feature 11 of the blast brain pool (FIGS. 2A-B feature 11), that was not pronounced in the control pool. Proteomic analysis determined that the dominant protein in this immunoreactive feature was, indeed, porcine IgG as opposed to another protein that had reacted with the 6B3 primary antibody. The increased presence of IgG in injured cortex was further verified by using a separate detection antibody specific to porcine IgG (FIGS. 4A-C), suggesting that an adaptive immune response to blast injury may have occurred in these animals. An analysis of the individual samples making up the pools of naive and blast-injured brain tissue further confirmed that blast injury was associated with significantly elevated levels of IgG in the cerebral cortex. The Coomassie-stained protein profile for each sample is presented in FIG. 4A. The corresponding western blot for porcine IgG is shown in FIG. 4B. FIG. 4C represents an integration analysis of the western blot signal intensities for IgG heavy and light chains (FIG. 4B), standardized to total protein load (FIG. 4A). The data show that blast significantly increased the amount of IgG detected in the cerebral cortex of blast-exposed swine. Variations in this response were observed among subjects, possibly reflecting variations in the degree of injury caused by the blast exposure.

This study did not account for individual differences and the likelihood that blast injury effects may vary across animals and brain regions, and a potential for animal variation is suggested by differences in the presence of IgGs, possibly autoantibodies, in the brain samples from individual animals (FIGS. 4A-C). Nevertheless, the results identified six specific proteins and their respective epitopes that are associated with blast-induced TBI.

### Example 2. Identification of Markers of Traumatic Brain Injury in Humans

This example investigates the effects of severe trauma on the deimination of proteins in human brain.

### Background:

Deimination is a posttranslational protein modification involving the enzymatic conversion of intrapeptidyl arginine to citrulline. Abnormal protein deimination is an underlying mechanism in the autoimmune diseases rheumatoid arthritis (RA) and multiple sclerosis (MS), where aberrant deimination creates antigenic epitopes that elicit sustained autoimmune attacks. This investigation examined the hypothesis that aberrant deimination of brain proteins occurs as a result of traumatic brain injury (TBI) and may thus contribute to long-term neuropathologies via autoimmune mechanisms. The present investigation identified 90 deiminated proteins in the cerebral cortex of young adult males who died as a result of traumatic motor vehicle accidents.

Oxygen deficiency and the resulting oxidative stress and calcium excitotoxicity are hallmarks of TBI which create a cellular environment that promotes aberrant protein deimination. Protein deimination is mediated by a small family of calcium-dependent enzymes, peptidylarginine deiminase (PAD), which is expressed in a tissue specific and developmentally regulated fashion and exhibits a selective subcellular localization. While most is known about the role of PAD in the nucleus, where it is involved in epigenetic regulation of gene expression via the deimination of histones, PAD is also present in the cytosol where its functions are not well understood. We previously observed in animal models that blast and controlled cortical impact TBI alters the deimination status of proteins in the brains of swine and rats, respectively. The affected proteins represent a relatively small subset of the entire brain proteome and include glial fibrillary acidic protein (GFAP) and vimentin, proteins known to be involved in autoimmune-based pathologies. This research has also revealed that injury was associated with an increase in immunoglobulins in the brain, possibly representing autoantibodies directed against novel protein epitopes. The present study extended these animal findings into humans. This investigation has defined a human TBI deiminome that can now serve as the basis for autoimmune profiling in TBI. Ultimately, autoimmune profiling may guide the use of immune therapies for treating chronic consequences of TBI, and importantly, provide justification for the acute use of small molecule inhibitors of PAD to prevent aberrant deimination and subsequent autoimmune reactions.

Subject data:

| | |
|---|---|
| N = 10 | |
| Ave Age = 26 y | range = 21-31 y |
| Ave Post Mortem Delay = 2.3 h | range = 2.5-4.25 h |

### Sample Preparation and Analysis:

Cerebral cortex were collected from 10 adult males (21-31 y, average 26 y) who died as a result of traumatic motor vehicle accidents (post mortem delay=2.0-4.25 h, average 2.32 h).

Tissues (1-2 gm each) were extracted in 5× volumes of 50 mM TRIS, pH 7.7 containing 1% CHAPS, protease inhibitors and EDTA. Aliquots of each extract were combine to produce a single pooled sample. Following centrifugation, the sample underwent spin filtration with volume replenishment to reduce the concentration for CHAPS to 0.1%. The sample was fractionated by fluid phase isoelectric focusing (IEF) to simplify the proteome. IEF fractions were analyzed for protein-bound citrulline by western blotting. Positive bands were mapped to corresponding features visualized by Coomassie staining of protein gels that were run in parallel. Bands were collected, digested with Lys-C and analyzed by tandem mass spectrometry by the Keck Lab, Yale University (ThermoFisher Orbitrap LC-MS/MS). Fragmentation spectra were analyzed by both the Mascot and Byonics search algorithms for the signature 43 Dalton neutral loss ion that is unique to a deiminated arginine.

### Criteria for Identification of a Deiminated Arginine:

### Mascot: Expectation Value of <0.5

Byonics: Mass error range of −3 to +3

### Log Probability of <1.3

### DeltaMod of <10

### Results:

A total of 90 proteins were identified as being deiminated, and of a total of 147 deimination sites were identified in the 90 proteins. The types of proteins most highly represented were those forming the cytoskeleton and involved in axonal growth, and mitochondrial enzymes. The list of proteins and deimination sites are shown in Tables 1-3.

### Example 3. Development of a Multiplex Platform for Detecting Anti-Deimination Antibodies in Response to Brain Trauma

This example describes an exemplary method for developing a multiplex platform for detecting antibodies specific to deimination proteins as described herein. With the identification of proteins that are deiminated in response to brain trauma, deiminated peptides may be produced. The deiminated peptides can be synthesized based on the deiminated arginine residue(s) identified in Example 2, and set forth in Tables 1-3.

For illustration, FIG. 8 shows the protein sequence for Glutathione reductase, mitochondrial isoform 1 precursor (accession number: NP_000628.2) with the two identified deiminated arginine residues shown in boxed, lowercase “r”. The two identified deiminated arginine residues correspond to R153 and R268 of the published sequence for accession number: NP_000628.2. As shown in FIG. 8, a deiminated peptide (e.g., peptides 801-806) may comprise an identified deiminated arginine residue and flanking amino acid residues. Alternatively, as also shown in FIG. 8, a deiminated peptide (e.g., peptides 807-809) may be based on an arginine residue that has not been confirmed as a deiminated arginine residue, but is a possible deiminated arginine residue (see circled, uppercase “R”). In such embodiments, the deiminated peptide (e.g., peptides 807-809) may comprise the possible deiminated arginine residue and flanking amino acid residues.

The deiminated peptides can be synthesized directly onto an array. For example, a peptide array can be generated based on the principle of in situ photolithographic synthesis using a microscope slide as a solid support. The microscope slide can be amino-functionalized with 6-amino hexanoic acid as a spacer and amino acid derivatives carrying a photosensitive 2-(2-nitrophenyl)propyl-oxycarbonyl group (NPPOC) at the a-amino function. Amino acids can be coupled to the array by preactivation of 30 mM amino acid, 30 mM 1-hydroxy-benzoatriazole/2-(1H-benzotriazole-1-yl)-1,1,3,3-tetramethylamium hexafluorphosphate, and 60 mM ethyl-diispropylamine, in dimethylformamide for a total of 5-7 minutes before flushing the substrate for 5 minutes. The substrate can be washed with N-methyl-2-pyrrolidone (NMP), and site-specific cleavage of the NPPOC group can be effected by irradiation of an image created by a Digital Micromirror Device (Texas Instruments, SXGA+ graphics format), projecting light at 365 nm wavelength. Final treatment of the slide with trifluoroacetic acid/water/triisopropylsilane for 30 min will remove the sidechain protection of the amino acids.

A peptide array comprising deiminated peptides as described herein may be used to detect antibodies specific to deiminated proteins described herein that are deiminated in response to traumatic brain injury or to diagnose and/or monitor traumatic brain injury in a subject.

### Example 4. Detection of Antibodies

This example describes an exemplary method for detecting antibodies specific to deiminated proteins that are deiminated in response to traumatic brain injury. A schematic of this exemplary method is depicted in FIG. 7. As shown in FIG. 7, a biological sample (701) comprising human IgG antibodies specific to deiminated proteins (referred to herein as autoantibodies) is applied to a peptide array (703). The peptide array comprises a plurality of different deiminated peptides (704). A wash buffer is applied to the peptide array to remove any antibodies that did not specifically bind to a deiminated peptide. Secondary antibodies (705), such as goat anti-human IgG antibodies, are applied to the peptide array. The secondary antibodies are optionally conjugated to a detectable label (706). A wash buffer is applied to the peptide array to remove any secondary antibodies that did not bind to the human IgG antibodies. The human IgG antibodies bound to the peptide array are detected, such as by using electrochemiluminescence, and the identity of the human IgG antibodies is determined based on the location of the electrochemiluminescent signal on the peptide array.

TABLE 1
Deiminated Proteins and Peptides
(Human)

| Protein Name | Peptide Sequence | Deiminated | SEQ ID |
|---|---|---|---|
| (accession no.) | (amino acid residues)* | Residue | NO: |
| 4-aminobutyrate | K.LrQSLLSVAPK.G (143-153) | R144 | 1 |
| aminotransferase, | K.ENQQEEArCLEEVEDLIVK.Y (261-279) | R268 | 2 |
| mitochondrial precursor | | | |
| (GABA transaminase) | | | |
| (NP_001120920.1) | | | |
| acetyl-CoA | K.MNAGSDPVVIVSAArTIIGSFNGALA | R115 | 3 |
| acetyltransferase, | AVPVQDLGSTVIK.E (1-39) | | |
| cytosolic isoform 1 | K.EIVPVLVSTrK.G (201-211) | R210 | 4 |
| (NP_005882.2) | | | |
| Aldehyde dehydrogenase | K.LLCGGGIAADrGYFIQPTVFGDVQDG | R394 | 5 |
| (NP_001677.2) | MTIAK.E (384-414) | | |
| | K.TIEEVVGrANNSTYGLAAAVFTK.D | R436 | 6 |
| | (429-451) | | |
| ATP synthase subunit | K.IPVGPETLGrIMNVIGEPIDErGPIK.T | R143 | 7 |
| beta (NP_001677.2) | (134-159) | R155 | |
| | K.IQrFLSQPFQVAEVFTGHMGK.L (460-480) | R462 | 8 |
| Contactin (CAA79696) | K.WrMNNGDVDLTSDRYSMVGGNLVI | R78 | 9 |
| | NNPDK.Q (77-105) | | |
| | K.rRFVSQTNGNLYIANVEASDK.G | R186 | 10 |
| | (186-206) | | |
| | K.VSLNCrARASPFPVYK.W (60-76) | R65 | 11 |
| Glyceraldehyde-3- | K.LWrDGRGALQNIIPASTGAAK.A | R197 | 12 |
| phosphate dehydrogenase | (195-215) | | |
| (AAH01601) | K.LTGMAFRVPTANVSVVDLTCrLEK.P | R248 | 13 |
| | (228-251) | | |
| Methylmalonate- | K.NLrNNAGDQPGADLGPLITPQAK.E | R344 | 14 |
| semialdehyde | (342-364) | | |
| dehydrogenase | K.ErVCNLIDSGTK.E (365-376) | R366 | 15 |
| (AAF04489) | | | |
| Microtubule-associated | K.SrLQTAPVPMPDLK.N (183-196) | R184 | 16 |
| protein tau (AAI14949.1) | | | |
| Phosphatidylethanolamine | K.NrPTSISWDGLDSGK.L (48-62) | R49 | 17 |
| binding protein | K.LYTLVLTDPDAPSrK.D (63-77) | R76 | 18 |
| (GenBank: CAA53031.1) | K.YELrAPVAGTCYQAEWDDYVPK.L | R161 | 19 |
| | (158-179) | | |
| Phosphoglycerate kinase | K.NNQITNNQrIK.A (31-41) | R39 | 20 |
| 1 (NP_000282.1) | K.SVVLMSHLGrPDGVPMPDK.Y (57-75) | R66 | 21 |
| | K.ALESPErPFLAILGGAK.V (200-216) | R206 | 22 |
| | K.ATSrGCITIIGGGDTATCCAK.W (362-382) | R365 | 23 |
| Phosphoserine | K.AGrCADYVVTGAWSAK.A (95-110) | R97 | 24 |
| aminotransferase | K.SQTIYEIIDNSQGFYVCPVEPQNrSK.M | R298 | 25 |
| (AAN71736.1) | (275-300) | | |
| Pyruvate dehydrogenase | K.CDLHrLEEGPPVTTVLTREDGLK.Y | R45 | 26 |
| E1 component subunit | (41-63) | | |
| alpha, somatic form, | | | |
| mitochondrial, isoform 1 | | | |
| and 2 (NP_000275.1 and | K.DrMVNSNLASVEELK.E (322-336) | R323 | 27 |
| NP_001166926.1) | | | |
| Retinal dehydrogenase 1 | K.TLrYCAGWADK.I (129-139) | R131 | 28 |
| (NP_000680.2) | K.rANNTFYGLSAGVFTK.D (420-435) | R420 | 29 |
| | K.MSGNGrELGEYGFHEYTEVK.T (471-490) | R476 | 30 |
| Transgelin-2 (GenBank: | K.ENPrNFSDNQLQEGK.N (157-171) | R160 | 31 |
| AAH09357.1) | | | |
| Ubiquitin carboxyl- | K.CFEKNEAIQAAHDAVAQEGQCrVDD | R153 | 32 |
| terminal hydrolase | K.V (132-157) | | |
| isozyme L1 | | | |
| (NP_004172.2) | | | |
| Ubiquitin-like modifier- | K.RLQTSSVLVSGLrGLGVEIAK.N (69-89) | R81 | 33 |
| activating enzyme | | | |
| (NP_695012.1) | | | |
| UTP-glucose-1- | K.rCEFVMEVTNK.T (275-285) | R275 | 34 |
| phosphate | K.IQrPPEDSIQPYEK.I (78-91) | R80 | 35 |
| uridylyltransferase | K.rLQEQNAIDMEIIVNAK.T (341-357) | R341 | 36 |
| (Swiss-Prot: Q16851.5) | | | |
| Acetyl-CoA | K.LNIArNEQDAYAINSYTRSK.A | R208 | 37 |
| acetyltransferase, | (204-223) | | |
| mitochondrial precursor | K.EAYMGNVLQGGEGQAPTrQQAVLG | R105 | 38 |
| (NP_000010.1) | AGLPISTPCTTINK.V (88-125) | | |
| Cytosolic acyl coenzyme | K.WrNGDIVQPVLNPEPNTVSYSQSSLI | R207 | 39 |
| A thioester hydrolase | HLVGPSDCTLHGFVHGGVMK.L | | |
| isoform hBACHb | (206-252) | | |
| (NP_863654.1) | K.LMDEVAGIVAArHCK.T (253-267) | R264 | 40 |
| Adenylate kinase | K.YGYTHLSTGDLLrSEVSSGSARGK.K | R44 | 41 |
| isoenzyme 1 isoform 1 | (32-55) | | |
| (NP_000467.1) | | | |
| Alpha-centractin | K.ErACYLSINPQK.D (219-230) | R220 | 42 |
| (NP_005727.1) | | | |
| ATP synthase subunit | K.AVDSLVPIGrGQRELIIGDRQTGK.T | R204 | 43 |
| alpha, mitochondrial | (195-218) | | |
| isoform a precursor | | | |
| (NP_001001937.1) | | | |
| Carbonyl reductase | K.TNFFGTrDVCTELLPLIK.P (113-130) | R119 | 44 |
| (GenBank: AAA52070.1) | | | |
| Carboxypeptidase Q | K.rTFEEIK.E (31-37) | R31 | 45 |
| preproprotein | | | |
| (NP_057218.1) | | | |
| Cysteine and glycine-rich | K.IGGSErCPRCSQAVYAAEK.V (113-131) | R118 | 46 |
| protein 1 (GenBank: | | | |
| AAH32493.1) | | | |
| Cytosol aminopeptidase | K.YrSAGACTAAAFLK.E (456-469) | R457 | 47 |
| (NP_056991.2) | | | |
| Dihydroplipoyl | K.NLGLEELGIELDPRGrIPVNTRFQTK.I | R336 | 48 |
| dehydrogenase | (321-346) | | |
| (dihydrolipoamide), | | | |
| mitochondrial, isoform 1, | | | |
| (NP_000099.2) | | | |
| Fascin (NP_003079.1) | K.VAFRDCEGrYLAPSGPSGTLK.A (221-241) | R229 | 49 |
| Flavin reductase | K.TVAGQDAVIVLLGTrNDLSPTTVMSE | R78 | 50 |
| (NADPH) | GARNIVAAMK.A (64-99) | | |
| (NP_000704.1) | K.RDAYVSLNAIYQNNLTK.S (147-164) | R153 | 51 |
| Glutathione reductase, | K.VLrSFDSMISTNCTEELENAGVEVLK.F | R268 | 52 |
| mitochondrial isoform 1 | (266-291) | | |
| precursor (NP_000628.2) | | | |
| Glutamine synthetase | K.VQAMYIWIDGTGEGLrCK.T (26-43) | R41 | 53 |
| (NP_001028216.1) | | | |
| Glutathione S-transferase | K.DQQEAALVDMVNDGVEDLrCK.Y | R103 | 54 |
| (GenBank: CAA30894.1) | (83-103) | | |
| Glutathione 5-transferase | K.AFMCrFEALEK.I (187-197) | R191 | 55 |
| Mu 3 (NP_000840.2) | | | |
| Heterogeneous nuclear | K.RAVSrEDSQRPGAHLTVK.K (88-105) | R92 | 56 |
| ribonucleoprotein A1-like | K.IFVGGIKEDTEEHHLrDYFEQYGK.I | R122 | 57 |
| 2 (NP_001011725.1) | (107-130) | | |
| Lamin isoform A | K.NIYSEELrETK.R (209-219) | R216 | 58 |
| (NP_733821.1) | | | |
| Neuronal pentraxin-1 | K.IDELErQVLSRVNTLEEGK.G (170-188) | R175 | 59 |
| precursor (NP_002513.2) | | | |
| Neurofilament light | K.TLEIEACrGMNEALEK.Q (316-331) | R323 | 60 |
| peptide (NP_006149.2) | | | |
| Phosphoglycerate kinase | K.ALENPVrPFLAILGGAK.V (200-216) | R206 | 61 |
| 2 (NP_620061.2) | | | |
| POTE ankyrin domain | K.IWHHTFYNELrVAPEEHPILLTEAPLN | R795 | 62 |
| family member E | PK.A (785-813) | | |
| (NP_001077007.1) | | | |
| Radixin isoform 1 | K.QLErAQLENEK.K (317-327) | R320 | 63 |
| (NP_001247422.1) | | | |
| Septin 2 (GenBank: | K.QQPTQFINPETPGYVGFANLPNQVHr | R29 | 64 |
| AAH14455.1) | KSVKK.G (4-34) | | |
| Septin-8 | K.AAVEALQSQALHATSQQPLrKDK.D | R422 | 65 |
| (UniProtKB/Swiss-Prot: | (403-425) | | |
| Q92599.4) | | | |
| Septin 11 (GenBank: | K.rNEFLGELQK.K (327-336) | R327 | 66 |
| AAH08083.3) | | | |
| Spectrin beta chain, non- | K.GEQVSQNGLPAEQGSPrMAETVDTS | R2140 | 67 |
| erythrocytic 1 isoform 1 | EMVNGATEQRTSS.K (2124-2161) | | |
| (NP_003119.2) | | | |
| Transcription elongation | K.PDIERErLK.A (157-165) | R163 | 68 |
| factor, mitochondrial | | | |
| (NP_078959.3) | | | |
| Vimentin (NP_003371.2) | K.VELQELNDrFANYIDK.V (105-120) | R113 | 69 |
| | K.FADLSEAANrNNDALRQAK.Q (295-313) | R304 | 70 |
| Annexin A7 isoform 1 | K.AGFGTDEQIVDVVANrSNDQRQK.I | R215 | 71 |
| (NP_001147.1) | (200-222) | | |
| Beta-adrenergic receptor | K.VPLVQrGSANGL (678-689) | R683 | 72 |
| kinase 1 (NP_001610.2) | | | |
| Cytosolic non-specific | K.LNrYNYIEGTK.M (451-461) | R453 | 73 |
| dipeptidase | | | |
| (UniProtKB/Swiss-Prot: | | | |
| Q96KP4.2) | | | |
| Endophillin-B2 isoform a | K.VPSrVTNGELLAQYMADAASELGPT | R87 | 74 |
| (NP_001273974.1) | TPYGK.T (84-113) | | |
| Heat shock 70 kDa | K.LLQDFFNGrDLNK.S (349-361) | R357 | 75 |
| protein 1B | | | |
| (NP_005337.2) | | | |
| Heat shock protein HSP | K.AQALrDNSTMGYMAAK.K (738-753) | R742 | 76 |
| 90-alpha isoform 1 | | | |
| (NP_001017963.2) | | | |
| Heat shock protein 105 | K.VEDVSAVEIVGGATrIPAVK.E (332-351) | R346 | 77 |
| kDa isoform 1 | | | |
| (NP_006635.2) | | | |
| Malate dehydrogenase, | K.AGAGSATLSMAYAGArFVFSLVDA | R257 | 78 |
| mitochondrial isoform1 | MNGK.E (242-269) | | |
| precursor (NP_005909.2) | | | |
| Mitogen-activated protein | M.AAAAAAGAGPEMVrGQVFDVGPrY | R15 | 79 |
| kinase 1 (NP_002736.3) | TNLSYIGEGAYGMVCSAYDNVNK.V | R24 | |
| | (2-48) | | |
| Neuronal-specific septin | K.IrQESMPFAVVGSDK.E (253-269) | R255 | 80 |
| 3 isoform A | | | |
| (NP_663786.2) | | | |
| Rab GDP dissociation | K.rKQNDVFGEAEQ. (436-447) | R436 | 81 |
| inhibitor alpha | | | |
| (NP_001484.1) | | | |
| Receptor-interacting | K.SrLFDTK.H (197-203) | R198 | 82 |
| serine/threonine-protein | | | |
| kinase 4 (NP_065690.2) | | | |
| Septin 4 (GenBank: | K.ALHQrVNIVPILAK.A (258-271) | R262 | 83 |
| AAH18056.3) | | | |
| Serotransferrin isoform 1 | K.TVrWCAVSEHEATK.C (24-37) | R26 | 84 |
| precursor (NP_001054.1) | | | |
| SRC kinase signaling | K.VVTPGASrLK.A (973-982) | R980 | 85 |
| inhibitor 1 | | | |
| (NP_938033.1) | | | |
| Synapsin-1 | K.SQSLTNAFNLPEPAPPrPSLSQDEVK.A | R679 | 86 |
| (UniProtKB/Swiss-Prot: | (663-688) | | |
| P17600.3) | | | |
| Succinate-semialdehyde | K.NLrVGNGFEEGTTQGPLINEK.A (380-400) | R382 | 87 |
| dehydrogenase, | | | |
| mitochrondrial isoform 1 | | | |
| (NP_733936.1) | | | |
| Tyrosine-protein | K.NArEITQDTNDITYADLNLPK.G (416-436) | R418 | 88 |
| phosphatase non-receptor | | | |
| type substrate 1 isoform 1 | | | |
| precursor | | | |
| (NP_001035111.1) | | | |
| Tubulin alpha-3C/D | K.EDAANNYArGHYTIGK.E (97-112) | R105 | 89 |
| chain (NP_525125.2) | | | |
| Ubiquitin-like modifier- | K.RLQTSSVLVSGLrGLGVEIAK.N (69-89) | R81 | 90 |
| activating enzyme 1 | | | |
| (NP_695012.1) | | | |
| V-type proton ATPase | K.YMrALDEYYDK.H (457-467) | R459 | 91 |
| catalytic subunit A | | | |
| (NP_001681.2) | | | |

*Amino acid residues based on amino acid sequence of protein accession number.
Lowercase “r” is the deiminated arginine residue.
+Deiminated arginine residue

TABLE 2
(Human)

| Protein Name | Peptide Sequence | Deiminated | SEQ ID |
|---|---|---|---|
| (accession no.) | (amino acid residues)* | Residue | NO: |
| 2′3′-cyclic-nucleotide 3′- | K.TLFILrGLPGSGK.S (51-63) | R56 | 92 |
| phosphodiesterase | K.ELrQFVPGDEPrEK.M (222-235) | R224 | 93 |
| isoform 1 (NP_149124.3) | | R233 | |
| | K.IFFrK.M (16-20) | R19 | 94 |
| | K.GGSrGEEVGELSRGK.L (357-371) | R360 | 95 |
| | K.TAWrLDCAQLK.E (153-161) | R156 | 96 |
| Creatine kinase B-type | K.EVFTrFCTGLTQIETLFK.S (248-265) | R252 | 97 |
| (NP_001814.2) | K.LrFPAEDEFPDLSAHNNHMAK.V | R13 | 98 |
| | (12-32) | | |
| | K.DLFDPIIEDrHGGYKPSDEHK.T | R96 | 99 |
| | (87-107) | | |
| | K.RGTGGVDTAAVGGVFDVSNADr | R341 | 100 |
| | LGFSEVELVQMVVDGVK.L (320-358) | | |
| Dihydropyrimidinase- | K.DNFTLIPEGTNGTEErMSVIWDK. | R466 | 101 |
| related protein 2 isoform | A (451-473) | | |
| 1 (NP_001184222.1) | | | |
| Dihydropyrimidinase- | K.NIPrITSDrLLIK.G (8-20) | R11 | 102 |
| related protein 2 isoform | | R16 | |
| 2 (NP_001377.1) | K.THNSSLEYNIFEGMECrGSPLVVIS | R440 | 103 |
| | QGK.I (424-451) | | |
| Fructose biphosphate | K.ELSDIALrIVAPGK.G (15-28) | R22 | 104 |
| aldolase C (ALDOC) | K.DDNGVPFVrTIQDK.G (88-101) | R96 | 105 |
| (GenBank: CAA30270.1) | K.GVVPLAGTDGETTTQGLDGLSEr | R134 | 106 |
| | CAQYK.K (112-139) | | |
| | K.DGADFAKWrCVLK.I (141-153) | R149 | 107 |
| | KISErTPSALAILENANVLArYASIC | R157 | 108 |
| | QQNGIVPIVEPEILPDGDHDLK.R | R173 | |
| | (154-200) | | |
| | K.rAEVNGLAAQGK.Y (331-342) | R331 | 109 |
| Gamma-enolase | K.AGAAERELPLYrHIAQLAGNSDLI | R132 | 110 |
| (NP_001966.1) | LPVPAFNVINGGSHAGNK.L (121-162) | | |
| Glial fibrillary acidic | K.EIrTQYEAMASSNMHEAEEWYRS | R239 | 111 |
| protein isoform 1 | K.F (237-260) | | |
| (NP_002046) | K.DEMArHLQEYQDLLNVK.L (340-356) | R344 | 112 |
| Gamma-Enolase | K.AGAAERELPLYrHIAQLAGNSDLI | R132 | 113 |
| (GenBank: AAB59554.1) | LPVPAFNVINGGSHAGNK.L (121-162) | | |
| 78 kDa glucose-regulated | K.LIPrNTVVPTK.K (436-446) | R439 | 114 |
| protein precursor | | | |
| (NP_005338.1) | | | |
| Actin, cytoplasmic 1 | K.DLYANTVLSGGTTMYPGIADrMQ | R312 | 115 |
| (NP_001092.1) | K.E (292-315) | | |
| Alpha Enolase | K.YNQLLrIEEELGSK.A (407-420) | R412 | 116 |
| (Swiss/UniProtKB | | | |
| PO6733.2) | | | |
| Cofilin (GenBank: | K.DCrYALYDATYETK.E (79-92) | R81 | 117 |
| CAA64685.1) | | | |
| Ermin (GenBank: | K.ErITEQPLKEEEDEDRK.N (132-148) | R133 | 118 |
| ABC67251.1) | | | |
| Moesin (NP_002435.1) | K.EALLQASrDQK.K (401-411) | R408 | 119 |
| Oligodendrocyte-myelin | K.IPKQYrTKETTFGATLSK.D (303-320) | R308 | 120 |
| glycoprotein precursor | | | |
| (NP_002535.3) | | | |
| Peptidyl-prolyl cis-trans | K.EGMNIVEAMErFGSRNGK.T (133-152) | R144 | 121 |
| isomerase A isoform 1 | | | |
| (NP_066953.1) | | | |
| Synapsin II | K.MNQLLSRTPALSPQrPLTTQQPQS | R428 | 122 |
| (UniProtKB/Swiss-Prot: | GTLK.D (414-441) | | |
| Q92777.3) | | | |
| Syntaxin binding protein | K.YrAAHVFFTDSCPDALFNELVK.S | R100 | 123 |
| 1 (GenBank: | (99-120) | | |
| AAH15749.1) | K.LCrVEQDLAMGTDAEGEK.I (365-382) | R367 | 124 |
| Transgelin | K.DMAAVQrTLMALGSLAVTK.N | R128 | 125 |
| (NP_001001522.1) | (122-140) | | |
| Tubulin alpha-1B chain | K.EDAANNYArGHYTIGK.E (97-112) | R105 | 126 |
| (NP_006073.2) | K.VQrAVCMLSNTTAIAEAWARLD | R373 | 127 |
| | HK.F (371-394) | | |
| Tubulin polymerization- | K.TGGAVDrLTDTSRYTGSEIK.E | R124 | 128 |
| promoting protein family | (118-136) | | |
| member 3 | | | |
| (NP_057048.2) | | | |
| Elongation factor 1-alpha | K.NGQTrEHALLAYTLGVK.Q (130-146) | R134 | 129 |
| 2 (UniProtKB/Swiss- | | | |
| Prot: Q05639.1) | K.IGGIGTVPVGrVETGILRPGMVVT | R266 | 130 |
| | FAPVNITTEVK.S (256-290) | | |

*Amino acid residues based on amino acid sequence of protein accession number.
Lowercase “r” is the deiminated arginine residue.

TABLE 3
(Human)

| Protein Name | Peptide Sequence | Deiminated | |
|---|---|---|---|
| (accession no.) | (amino acid residues)* | Residue | SEQ ID NO: |
| 2′3′-cyclic-nucleotide | K.STLArVIVDK.Y (64-73) | R68 | 131 |
| 3′-phosphodiesterase | K.ITPGArGAFSEEYK.R (88-101) | R93 | 132 |
| isoform 1 | | | |
| (NP_149124.3) | | | |
| Glial fibrillary acidic | K.VrFLEQQNK.A (87-95) | R88 | 133 |
| protein isoform 1 | K.ALAAELNQLrAK.E (96-107) | R105 | 134 |
| (NP_002046) | K.FADLTDAAARNAELLrQAK.H | R276 | 135 |
| | (261-279) | | |
| | K.TVEMrDGEVIK.E (412-422) | R416 | 136 |
| Myelin basic protein | K.DSHHPArTAHYGSLPQK.S (86-102) | R92 | 137 |
| (AAC41944.1) | K.SHGrTQDENPVVHFFK.N (103-118) | R106 | 138 |
| | K.NIVTPrTPPPSQGK.G (119-132) | R124 | 139 |
| Tubulin polymerization- | K.AISSPTVSrLTDTTK.F (157-171) | R166 | 140 |
| promoting protein | | | |
| (NP_008961.1) | | | |
| Tubulin polymerization- | K.GIAGrQDILDDSGYVSAYK.N | R151 | 141 |
| promoting protein | (147-165) | | |
| family member 3 | | | |
| (NP_057048.2) | | | |
| Elongation factor 1- | K.PLrLPLQDVYK.I (245-55) | R247 | 142 |
| alpha 2 | | | |
| (UniProtKB/Swiss-Prot: | | | |
| Q05639.1) | | | |

*Amino acid residues based on amino acid sequence of protein accession number.
Lowercase “r” is the deiminated arginine residue.

TABLE 4
(Porcine)

| Protein Name | Peptide Sequence (amino acid residues)* | SEQ ID NO: |
|---|---|---|
| 4-aminobutyrate | LVQQPQNVSTFINrPALGILPPENFVEK | 143 |
| aminotransferase, | | |
| mitochondrial precursor | | |
| (GABA transaminase) | | |
| aconitase hydratase | LNrPTLSEK | 144 |
| Glial fibrillary acidic | ITIPVQTFSNLQIrETSLDTK | 145 |
| protein isoform 1 | TVEMrDGEVIK | 146 |
| Glutathione S-transferase | AFLASPEHVNrPINGNGK | 147 |
| histone H4 | SGLIYEETrGVLKVFLENVIRDAVTYTEHAK | 148 |
| Vimentin | TVETrDGQVINETSQHHDDLE | 149 |

*Lowercase “r” is the deiminated arginine residue.

TABLE 5
Miscellaneous Sequences

| Protein Name | Sequence | SEQ ID NO: |
|---|---|---|
| Glutathione | MALLPRALSAGAGPSWRRAARAFRGFLLLLPEPAALT | 150 |
| reductase, | RALSRAMACRQEPQPQGPPPAAGAVASYDYLVIGGGS | |
| mitochondrial | GGLASARRAAELGARAAVVESHKLGGTCVNVGCVPK | |
| isoform 1 | KVMWNTAVHSEFMHDHADYGFPSCEGKFNWRVIKEK | |
| precursor | RDAYVSrLNAIYQNNLTKSHIEIIRGHAAFTSDPKPTIEV | |
| (NP_000628.2) | SGKKYTAPHILIATGGMPSTPHESQIPGASLGITSDGFFQ | |
| | LEELPGRSVIVGAGYIAVEMAGILSALGSKTSLMIRHD | |
| | KVLrSFDSMISTNCTEELENAGVEVLKFSQVKEVKKTL | |
| | SGLEVSMVTAVPGRLPVMTMIPDVDCLLWAIGRVPNT | |
| | KDLSLNKLGIQTDDKGHIIVDEFQNTNVKGIYAVGDVC | |
| | GKALLTPVAIAAGRKLAHRLFEYKEDSKLDYNNIPTVV | |
| | FSHPPIGTVGLTEDEAIHKYGIENVKTYSTSFTPMYHAV | |
| | TKRKTKCVMKMVCANKEEKVVGIHMQGLGCDEMLQ | |
| | GFAVAVKMGATKADFDNTVAIHPTSSEELVTLR | |

Lowercase “r” is the deiminated arginine residue.

### CITED REFERENCES

1. Ansari M A, Roberts K N, Scheff S W. Oxidative stress and modification of synaptic proteins in hippocampus after traumatic brain injury. Free Radic Biol Med 2008,45:443-452.

2. Darwish R S, Amiridze N, Aarabi B. Nitrotyrosine as an oxidative stress marker: evidence for involvement in neurologic outcome in human traumatic brain injury. J Trauma 2007,63:439-442.

3. Goldstein L E, Fisher A M, Tagge C A, Zhang X L, Velisek L, Sullivan J A, et al. Chronic traumatic encephalopathy in blast-exposed military veterans and a blast neurotrauma mouse model. Sci Transl Med 2012,4:134ra160.

4. Opii W O, Nukala V N, Sultana R, Pandya J D, Day K M, Merchant M L, et al. Proteomic identification of oxidized mitochondrial proteins following experimental traumatic brain injury. J Neurotrauma 2007,24:772-789.

5. de Lanerolle N C, Bandak F, Kang D, Li AY, Du F, Swauger P, et al. Characteristics of an explosive blast-induced brain injury in an experimental model. J Neuropathol Exp Neurol 2011,70:1046-1057.

6. Shlosberg D, Benifla M, Kaufer D, Friedman A. Blood-brain barrier breakdown as a therapeutic target in traumatic brain injury. Nat Rev Neurol 2010,6:393-403.

7. Fineman I, Hovda D A, Smith M, Yoshino A, Becker D P. Concussive brain injury is associated with a prolonged accumulation of calcium: a 45Ca autoradiographic study. Brain Res 1993,624:94-102.

8. Sun D A, Deshpande L S, Sombati S, Baranova A, Wilson M S, Hamm R J, et al. Traumatic brain injury causes a long-lasting calcium (Ca2+)-plateau of elevated intracellular Ca levels and altered Ca2+ homeostatic mechanisms in hippocampal neurons surviving brain injury. Eur J Neurosci 2008,27:1659-1672.

9. Belanger H G, Proctor-Weber Z, Kretzmer T, Kim M, French L M, Vanderploeg R D. Symptom complaints following reports of blast versus non-blast mild TBI: does mechanism of injury matter? Clin Neuropsychol 2011,25:702-715.

10. Elder G A, Dorr N P, De Gasperi R, Gama Sosa M A, Shaughness M C, Maudlin-Jeronimo E, et al. Blast exposure induces post-traumatic stress disorder-related traits in a rat model of mild traumatic brain injury. J Neurotrauma 2012,29:2564-2575.

11. Levin H S, Wilde E, Troyanskaya M, Petersen N J, Scheibel R, Newsome M, et al. Diffusion tensor imaging of mild to moderate blast-related traumatic brain injury and its sequelae. J Neurotrauma 2010,27:683-694.

12. Schultz B A, Cifu D X, McNamee S, Nichols M, Came W. Assessment and treatment of common persistent sequelae following blast induced mild traumatic brain injury. NeuroRehabilitation 2011,28:309-320.

13. Vanderploeg R D, Belanger H G, Homer R D, Spehar A M, Powell-Cope G, Luther S L, et al. Health outcomes associated with military deployment: mild traumatic brain injury, blast, trauma, and combat associations in the Florida National Guard. Arch Phys Med Rehabil 2012,93:1887-1895.

14. Blennow K, Brody D L, Kochanek P M, Levin H, McKee A, Ribbers G M, et al. Traumatic brain injuries. Nat Rev Dis Primers 2016,2:16084.

15. Zetterberg H, Blennow K. Fluid biomarkers for mild traumatic brain injury and related conditions. Nat Rev Neurol 2016,12:563-574.

16. Iverson G L, Gardner A J, McCrory P, Zafonte R, Castellani R J. A critical review of chronic traumatic encephalopathy. Neurosci Biobehav Rev 2015,56:276-293.

17. Lucke-Wold B P, Turner R C, Logsdon A F, Bailes J E, Huber J D, Rosen C L. Linking traumatic brain injury to chronic traumatic encephalopathy: identification of potential mechanisms leading to neurofibrillary tangle development. J Neurotrauma 2014,31:1129-1138.

18. McKee A C, Cantu R C, Nowinski C J, Hedley-Whyte E T, Gavett B E, Budson A E, et al. Chronic traumatic encephalopathy in athletes: progressive tauopathy after repetitive head injury. J Neuropathol Exp Neurol 2009,68:709-735.

19. Bicker K L, Thompson P R. The protein arginine deiminases: Structure, function, inhibition, and disease. Biopolymers 2013,99:155-163.

20. Gyorgy B, Toth E, Tarcsa E, Falus A, Buzas E I. Deimination: a posttranslational modification in health and disease. Int J Biochem Cell Biot 2006,38:1662-1677.

21. Girbal-Neuhauser E, Durieux J J, Arnaud M, Dalbon P, Sebbag M, Vincent C, et al. The epitopes targeted by the rheumatoid arthritis-associated antifilaggrin autoantibodies are posttranslationally generated on various sites of (pro)filaggrin by deimination of arginine residues. J Immunol 1999,162:585-594.

22. Reyes-Castillo Z, Palafox-Sanchez C A, Parra-Rojas I, Martinez-Bonilla G E, del Toro-Arreola S, Ramirez-Duenas M G, et al. Comparative analysis of autoantibodies targeting peptidylarginine deiminase type 4, mutated citrullinated vimentin and cyclic citrullinated peptides in rheumatoid arthritis: associations with cytokine profiles, clinical and genetic features. Clin Exp Immunol 2015,182:119-131.

23. Schellekens G A, de Jong B A, van den Hoogen F H, van de Putte L B, van Venrooij W J. Citrulline is an essential constituent of antigenic determinants recognized by rheumatoid arthritis-specific autoantibodies. J Clin Invest 1998,101:273-281.

24. Mastrangelo A, Colasanti T, Barbati C, Pecani A, Sabatinelli D, Pendolino M, et al. The Role of Posttranslational Protein Modifications in Rheumatological Diseases: Focus on Rheumatoid Arthritis. 2015,2015:712490.

25. Bradford C M, Ramos I, Cross A K, Haddock G, McQuaid S, Nicholas A P, et al. Localisation of citrullinated proteins in normal appearing white matter and lesions in the central nervous system in multiple sclerosis. J Neuroimmunol 2014,273:85-95.

26. Nicholas A P, Sambandam T, Echols J D, Tourtellotte W W. Increased citrullinated glial fibrillary acidic protein in secondary progressive multiple sclerosis.J Comp Neurol 2004,473:128-136.

27. Stadelmann C. Multiple sclerosis as a neurodegenerative disease: pathology, mechanisms and therapeutic implications. Curr Opin Neurol 2011,24:224-229.

28. Yang L, Tan D, Piao H. Myelin Basic Protein Deimination in Multiple Sclerosis: A Potential Therapeutic Target for the Pathology. Neurochem Res 2016,41:1845-1856.

29. Kipnis J. Multifaceted interactions between adaptive immunity and the central nervous system. Science 2016,353:766-771.

30. McKee CA, Lukens JR. Emerging Roles for the Immune System in Traumatic Brain Injury. Front Immunol 2016,7:556.

31. Lazarus R C, Buonora J E, Flora M N, Freedy J G, Holstein G R, Martinelli G P, et al. Protein Deimination: A Proposed Mechanism for Pathology in Traumatic Brain Injury. Front Neurol 2015,6:204.

32. Hecker M, Fitzner B, Wendt M, Lorenz P, Flechtner K, Steinbeck F, et al. High-Density Peptide Microarray Analysis of IgG Autoantibody Reactivities in Serum and Cerebrospinal Fluid of Multiple Sclerosis Patients. Mol Cell Proteomics 2016,15:1360-1380.

33. Ishigami A, Masutomi H, Handa S, Nakamura M, Nakaya S, Uchida Y, et al. Mass spectrometric identification of deimination sites and immunohistochemical detection of citrullinated glial fibrillary acidic protein in Alzheimer's disease brains. J Neurosci Res 2015,93:1664-1674.

34. Jin Z, Fu Z, Yang J, Troncosco J, Everett A D, Van Eyk J E. Identification and characterization of citrulline-modified brain proteins by combining HCD and CID fragmentation. Proteomics 2013,13:2682-2691.

35. Ahmed F, Gyorgy A, Kamnaksh A, Ling G, Tong L, Parks S, et al. Time-dependent changes of protein biomarker levels in the cerebrospinal fluid after blast traumatic brain injury. Electrophoresis 2012,33:3705-3711.

36. Gyorgy A, Ling G, Wingo D, Walker J, Tong L, Parks S, et al. Time-dependent changes in serum biomarker levels after blast traumatic brain injury. J Neurotrauma 2011,28:1121-1126.

37. Hao G, Wang D, Gu J, Shen Q, Gross S S, Wang Y. Neutral loss of isocyanic acid in peptide CID spectra: a novel diagnostic marker for mass spectrometric identification of protein deimination. J Am Soc Mass Spectrom 2009,20:723-727.

38. Martinelli G P, Friedrich V L, Jr., Holstein G R. L-citrulline immunostaining identifies nitric oxide production sites within neurons. Neuroscience 2002,114:111-122.

39. Zhang Z, Zoltewicz J S, Mondello S, Newsom K J, Yang Z, Yang B, et al. Human traumatic brain injury induces autoantibody response against glial fibrillary acidic protein and its breakdown products. PLoS One 2014,9:e92698.

40. Ytterberg A J, Joshua V, Reynisdottir G, Tarasova N K, Rutishauser D, Ossipova E, et al. Shared immunological targets in the lungs and joints of patients with rheumatoid arthritis: identification and validation. Ann Rheum Dis 2015,74:1772-1777.

41. Aungst S L, Kabadi S V, Thompson S M, Stoica B A, Faden A I. Repeated mild traumatic brain injury causes chronic neuroinflammation, changes in hippocampal synaptic plasticity, and associated cognitive deficits. J Cereb Blood Flow Metab 2014,34:1223-1232.

42. Loane D J, Kumar A. Microglia in the TBI brain: The good, the bad, and the dysregulated. Exp Neurol 2016,275 Pt 3:316-327.

43. Marchi N, Bazarian J J, Puvenna V, Janigro M, Ghosh C, Zhong J, et al. Consequences of repeated blood-brain barrier disruption in football players. PLoS One 2013,8:e56805.

44. Gottenberg J E, Ravaud P, Cantagrel A, Combe B, Flipo R M, Schaeverbeke T, et al. Positivity for anti-cyclic citrullinated peptide is associated with a better response to abatacept: data from the ‘Orencia and Rheumatoid Arthritis’ registry. Ann Rheum Dis 2012,71:1815-1819.

45. Chatzidionysiou K, Lie E, Nasonov E, Lukina G, Hetland M L, Tarp U, et al. Highest clinical effectiveness of rituximab in autoantibody-positive patients with rheumatoid arthritis and in those for whom no more than one previous TNF antagonist has failed: pooled data from 10 European registries. Ann Rheum Dis 2011,70:1575-1580.

46. Lal P, Su Z, Holweg C T, Silverman G J, Schwartzman S, Kelman A, et al. Inflammation and autoantibody markers identify rheumatoid arthritis patients with enhanced clinical benefit following rituximab treatment. Arthritis Rheum 2011,63:3681-3691.

47. Harauz G, Musse A A. A tale of two citrullines-structural and functional aspects of myelin basic protein deimination in health and disease. Neurochem Res 2007,32:137-158.

48. Ishigami A, Ohsawa T, Hiratsuka M, Taguchi H, Kobayashi S, Saito Y, et al. Abnormal accumulation of citrullinated proteins catalyzed by peptidylarginine deiminase in hippocampal extracts from patients with Alzheimer's disease. J Neurosci Res 2005,80:120-128.

49. Jang B, Ishigami A, Maruyama N, Carp R I, Kim Y S, Choi E K. Peptidylarginine deiminase and protein deimination in prion diseases: strong evidence of neurodegeneration. Prion 2013,7:42-46.

50. Louw C, Gordon A, Johnston N, Mollatt C, Bradley G, Whiteley C G. Arginine deiminases: therapeutic tools in the etiology and pathogenesis of Alzheimer's disease. J Enzyme Inhib Med Chem 2007,22:121-126.

1-57. (canceled)

58. A method of diagnosing oxygen-deprivation brain injury (ODBI) or oxygen-deprivation causing injury (ODCI) in a subject, comprising:
(a) analyzing a biological sample taken from the subject to detect antibodies specific to a deiminated variant of one or more of the proteins listed in Table 1, optionally to detect antibodies specific to a deiminated variant of one or more of the proteins listed in Table 2 or a fragment thereof, optionally wherein the deiminated variant is deiminated at a site(s) listed in Table 2, or a fragment thereof, and/or optionally to detect antibodies specific to a deiminated variant of one or more of the proteins listed in Table 3 or a fragment thereof, optionally wherein the deiminated variant is deiminated at a site(s) listed in Table 3, or a fragment thereof; and
(b) comparing the detected antibody level(s) to reference antibody level(s) of antibodies specific to a deiminated variant of the same protein(s) in a reference sample,
wherein an increase in the detected antibody level(s) as compared to the reference antibody level(s) is indicative that the subject is suffering from ODBI or ODCI.

59. The method of claim 58, wherein the reference sample is a sample previously obtained from the subject.

60. The method of claim 58, wherein the reference sample is a sample from one or more reference subjects determined not to have ODBI or ODCI.

61. The method of claim 58, wherein the antibodies are detected by contacting the biological sample(s) with a deiminated peptide that comprises at least a deiminated portion of the deiminated variant.

62. The method of claim 61, wherein the deiminated peptide is deiminated at a site listed in any one of Table 1, Table 2, and Table 3.

63. The method of claim 61, wherein the deiminated peptide comprise from 3 to 25 amino acid residues.

64. The method of claim 58, wherein the biological sample(s) is selected from blood, cerebrospinal fluid (CSF), urine, saliva, stool, and synovial fluid.

65. The method of claim 58, wherein the biological sample is blood.

66. A method of determining the efficacy of an ODBI-therapy or ODCI-therapy in a subject being treated with the ODBI-therapy or ODCI-therapy, comprising:
(a) applying a first biological sample taken from the subject to a first device comprising a plurality of deiminated peptides to detect antibody level(s) in the first biological sample, wherein at least one deiminated peptide comprises a deiminated portion of a deiminated variant of a protein listed in Table 1, optionally wherein at least one deiminated peptide comprises a deiminated portion of a deiminated variant of a protein listed in Table 2, and/or optionally wherein at least one deiminated peptide comprises a deiminated portion of a deiminated variant of a protein listed in Table 3; and
(b) applying a second biological sample taken from the subject at a time subsequent to the first biological sample to a second device comprising a plurality of deiminated peptides to detect antibody level(s) in the second biological sample, wherein at least one deiminated peptide comprises a deiminated portion of a deiminated variant of a protein listed in Table 1, optionally wherein at least one deiminated peptide comprises a deiminated portion of a deiminated variant of a protein listed in Table 2, and/or optionally wherein at least one deiminated peptide comprises a deiminated portion of a deiminated variant of a protein listed in Table 3; and
(c) comparing the detected antibody level(s) in the first and second biological samples, wherein an increase in the detected antibody level(s) in the second biological sample relative to the first biological sample is indicative that the therapy is not effective, and/or wherein a decrease in the detected antibody level(s) in the second biological sample relative to the first biological sample is indicative that the therapy is effective.

67. The method of claim 66, wherein the subject is administered the ODBI-therapy or ODCI-therapy prior to the second biological sample being taken from the subject.

68. The method of claim 66, wherein the ODBI-therapy and/or ODCI-therapy comprises one or more anti-immune drugs and/or anti-idiotypic antibodies.

69. The method of claim 66, wherein the antibodies are detected by contacting the biological sample(s) with a deiminated peptide that comprises at least a deiminated portion of the deiminated variant.

70. The method of claim 69, wherein the deiminated peptide is deiminated at a site listed in any one of Table 1, Table 2, and Table 3.

71. The method of claim 69, wherein the deiminated peptide comprise from 3 to 25 amino acid residues.

72. The method of claim 66, wherein the first and second biological samples are selected from blood, cerebrospinal fluid (CSF), urine, saliva, stool, and synovial fluid.

73. The method of claim 66, wherein the first and second biological samples are blood.

74. A device comprising a plurality of deiminated peptides, wherein at least one deiminated peptide is selected from (a) a demininated peptide that comprises a deiminated portion of a deiminated variant of a protein listed in Table 1 and (a) a deiminated peptide that comprises a deiminated portion of a deiminated variant of a protein listed in Table 2 that is deiminated at a site listed in Table 2.

75. The device of claim 74, further comprising one or more deiminated peptides comprising a deiminated portion of a deiminated variant of one or more proteins listed in Tables 1-3 or a fragment thereof.

76. A method for treating an ODBI or ODCI in a subject in need thereof, comprising administering to the subject a deiminase inhibitor, optionally wherein the deiminase inhibitor inhibits or suppresses the activity of a protein arginine deiminase (PAD), optionally wherein the PAD is selected from PAD 1, PAD 2, PAD 3, PAD 4, and PAD 6, optionally wherein the deiminase inhibitor is selected from taxol, minocycline, streptomycin, o-F-amidine, o-Cl-amidine, Thr-Asp-F-amidine (TDFA), thioredoxin, streptonigrin, and analogs and derivatives thereof.


SEQUENCE LISTING (152 sequences)

SEQ ID NO: 1 - protein, 13 aa, Homo sapiens
  MOD_RES (3)..(3): Deiminated arginine residue
KLXQSLLSVAPKG

SEQ ID NO: 2 - protein, 21 aa, Homo sapiens
  MOD_RES (9)..(9): Deiminated arginine residue
KENQQEEAXCLEEVEDLIVKY

SEQ ID NO: 3 - protein, 41 aa, Homo sapiens
  MOD_RES (16)..(16): Deiminated arginine residue
KMNAGSDPVVIVSAAXTIIGSFNGALAAVPVQDLGSTVIKE

SEQ ID NO: 4 - protein, 13 aa, Homo sapiens
  MOD_RES (11)..(11): Deiminated arginine residue
KEIVPVLVSTXKG

SEQ ID NO: 5 - protein, 33 aa, Homo sapiens
  MOD_RES (12)..(12): Deiminated arginine residue
KLLCGGGIAADXGYFIQPTVFGDVQDGMTIAKE

SEQ ID NO: 6 - protein, 25 aa, Homo sapiens
  MOD_RES (9)..(9): Deiminated arginine residue
KTIEEVVGXANNSTYGLAAAVFTKD

SEQ ID NO: 7 - protein, 28 aa, Homo sapiens
  MOD_RES (11)..(11): Deiminated arginine residue
  MOD_RES (23)..(23): Deiminated arginine residue
KIPVGPETLGXIMNVIGEPIDEXGPIKT

SEQ ID NO: 8 - protein, 23 aa, Homo sapiens
  MOD_RES (4)..(4): Deiminated arginine residue
KIQXFLSQPFQVAEVFTGHMGKL

SEQ ID NO: 9 - protein, 31 aa, Homo sapiens
  MOD_RES (3)..(3): Deiminated arginine residue
KWXMNNGDVDLTSDRYSMVGGNLVINNPDKQ

SEQ ID NO: 10 - protein, 23 aa, Homo sapiens
  MOD_RES (2)..(2): Deiminated arginine residue
KXRFVSQTNGNLYIANVEASDKG

SEQ ID NO: 11 - protein, 18 aa, Homo sapiens
  MOD_RES (7)..(7): Deiminated arginine residue
KVSLNCXARASPFPVYKW

SEQ ID NO: 12 - protein, 23 aa, Homo sapiens
  MOD_RES (4)..(4): Deiminated arginine residue
KLWXDGRGALQNIIPASTGAAKA

SEQ ID NO: 13 - protein, 26 aa, Homo sapiens
  MOD_RES (22)..(22): Deiminated arginine residue
KLTGMAFRVPTANVSVVDLTCXLEKP

SEQ ID NO: 14 - protein, 25 aa, Homo sapiens
  MOD_RES (4)..(4): Deiminated arginine residue
KNLXVNAGDQPGADLGPLITPQAKE

SEQ ID NO: 15 - protein, 14 aa, Homo sapiens
  MOD_RES (3)..(3): Deiminated arginine residue
KEXVCNLIDSGTKE

SEQ ID NO: 16 - protein, 16 aa, Homo sapiens
  MOD_RES (3)..(3): Deiminated arginine residue
KSXLQTAPVPMPDLKN

SEQ ID NO: 17 - protein, 17 aa, Homo sapiens
  MOD_RES (3)..(3): Deiminated arginine residue
KNXPTSISWDGLDSGKL

SEQ ID NO: 18 - protein, 17 aa, Homo sapiens
  MOD_RES (15)..(15): Deiminated arginine residue
KLYTLVLTDPDAPSXKD

SEQ ID NO: 19 - protein, 24 aa, Homo sapiens
  MOD_RES (5)..(5): Deiminated arginine residue
KYELXAPVAGTCYQAEWDDYVPKL

SEQ ID NO: 20 - protein, 13 aa, Homo sapiens
  MOD_RES (10)..(10): Deiminated arginine residue
KNNQITNNQXIKA

SEQ ID NO: 21 - protein, 21 aa, Homo sapiens
  MOD_RES (11)..(11): Deiminated arginine residue
KSVVLMSHLGXPDGVPMPDKY

SEQ ID NO: 22 - protein, 19 aa, Homo sapiens
  MOD_RES (8)..(8): Deiminated arginine residue
KALESPEXPFLAILGGAKV

SEQ ID NO: 23 - protein, 23 aa, Homo sapiens
  MOD_RES (5)..(5): Deiminated arginine residue
KATSXGCITIIGGGDTATCCAKW

SEQ ID NO: 24 - protein, 18 aa, Homo sapiens
  MOD_RES (4)..(4): Deiminated arginine residue
KAGXCADYVVTGAWSAKA

SEQ ID NO: 25 - protein, 28 aa, Homo sapiens
  MOD_RES (25)..(25): Deiminated arginine residue
KSQTIYEIIDNSQGFYVCPVEPQNXSKM

SEQ ID NO: 26 - protein, 25 aa, Homo sapiens
  MOD_RES (6)..(6): Deiminated arginine residue
KCDLHXLEEGPPVTTVLTREDGLKY

SEQ ID NO: 27 - protein, 17 aa, Homo sapiens
  MOD_RES (3)..(3): Deiminated arginine residue
KDXMVNSNLASVEELKE

SEQ ID NO: 28 - protein, 13 aa, Homo sapiens
  MOD_RES (4)..(4): Deiminated arginine residue
KTLXYCAGWADKI

SEQ ID NO: 29 - protein, 18 aa, Homo sapiens
  MOD_RES (2)..(2): Deiminated arginine residue
KXANNTFYGLSAGVFTKD

SEQ ID NO: 30 - protein, 22 aa, Homo sapiens
  MOD_RES (7)..(7): Deiminated arginine residue
KMSGNGXELGEYGFHEYTEVKT

SEQ ID NO: 31 - protein, 17 aa, Homo sapiens
  MOD_RES (5)..(5): Deiminated arginine residue
KENPXNFSDNQLQEGKN

SEQ ID NO: 32 - protein, 28 aa, Homo sapiens
  MOD_RES (23)..(23): Deiminated arginine residue
KCFEKNEAIQAAHDAVAQEGQCXVDDKV

SEQ ID NO: 33 - protein, 23 aa, Homo sapiens
  MOD_RES (14)..(14): Deiminated arginine residue
KRLQTSSVLVSGLXGLGVEIAKN

SEQ ID NO: 34 - protein, 13 aa, Homo sapiens
  MOD_RES (2)..(2): Deiminated arginine residue
KXCEFVMEVTNKT

SEQ ID NO: 35 - protein, 16 aa, Homo sapiens
  MOD_RES (4)..(4): Deiminated arginine residue
KIQXPPEDSIQPYEKI

SEQ ID NO: 36 - protein, 19 aa, Homo sapiens
  MOD_RES (2)..(2): Deiminated arginine residue
KXLQEQNAIDMEIIVNAKT

SEQ ID NO: 37 - protein, 22 aa, Homo sapiens
  MOD_RES (6)..(6): Deiminated arginine residue
KLNIAXNEQDAYAINSYTRSKA

SEQ ID NO: 38 - protein, 40 aa, Homo sapiens
  MOD_RES (19)..(19): Deiminated arginine residue
KEAYMGNVLQGGEGQAPTXQQAVLGAGLPISTPCTTINKV

SEQ ID NO: 39 - protein, 48 aa, Homo sapiens
  MOD_RES (3)..(3): Deiminated arginine residue
KWXNGDIVQPVLNPEPNTVSYSQSSLIHLVGPSDCTLHGFVHGGVMKL

SEQ ID NO: 40 - protein, 17 aa, Homo sapiens
  MOD_RES (13)..(13): Deiminated arginine residue
KLMDEVAGIVAAXHCKT

SEQ ID NO: 41 - protein, 26 aa, Homo sapiens
  MOD_RES (14)..(14): Deiminated arginine residue
KYGYTHLSTGDLLXSEVSSGSARGKK

SEQ ID NO: 42 - protein, 14 aa, Homo sapiens
  MOD_RES (3)..(3): Deiminated arginine residue
KEXACYLSINPQKD

SEQ ID NO: 43 - protein, 26 aa, Homo sapiens
  MOD_RES (11)..(11): Deiminated arginine residue
KAVDSLVPIGXGQRELIIGDRQTGKT

SEQ ID NO: 44 - protein, 20 aa, Homo sapiens
  MOD_RES (8)..(8): Deiminated arginine residue
KTNFFGTXDVCTELLPLIKP

SEQ ID NO: 45 - protein, 9 aa, Homo sapiens
  MOD_RES (2)..(2): Deiminated arginine residue
KXTFEEIKE

SEQ ID NO: 46 - protein, 21 aa, Homo sapiens
  MOD_RES (7)..(7): Deiminated arginine residue
KIGGSEXCPRCSQAVYAAEKV

SEQ ID NO: 47 - protein, 16 aa, Homo sapiens
  MOD_RES (3)..(3): Deiminated arginine residue
KYXSAGACTAAAFLKE

SEQ ID NO: 48 - protein, 28 aa, Homo sapiens
  MOD_RES (17)..(17): Deiminated arginine residue
KNLGLEELGIELDPRGXIPVNTRFQTKI

SEQ ID NO: 49 - protein, 23 aa, Homo sapiens
  MOD_RES (10)..(10): Deiminated arginine residue
KVAFRDCEGXYLAPSGPSGTLKA

SEQ ID NO: 50 - protein, 38 aa, Homo sapiens
  MOD_RES (16)..(16): Deiminated arginine residue
KTVAGQDAVIVLLGTXNDLSPTTVMSEGARNIVAAMKA

SEQ ID NO: 51 - protein, 20 aa, Homo sapiens
  MOD_RES (8)..(8): Deiminated arginine residue
KRDAYVSXLNAIYQNNLTKS

SEQ ID NO: 52 - protein, 28 aa, Homo sapiens
  MOD_RES (4)..(4): Deiminated arginine residue
KVLXSFDSMISTNCTEELENAGVEVLKF

SEQ ID NO: 53 - protein, 20 aa, Homo sapiens
  MOD_RES (17)..(17): Deiminated arginine residue
KVQAMYIWIDGTGEGLXCKT

SEQ ID NO: 54 - protein, 23 aa, Homo sapiens
  MOD_RES (20)..(20): Deiminated arginine residue
KDQQEAALVDMVNDGVEDLXCKY

SEQ ID NO: 55 - protein, 13 aa, Homo sapiens
  MOD_RES (6)..(6): Deiminated arginine residue
KAFMCXFEALEKI

SEQ ID NO: 56 - protein, 20 aa, Homo sapiens
  MOD_RES (6)..(6): Deiminated arginine residue
KRAVSXEDSQRPGAHLTVKK

SEQ ID NO: 57 - protein, 26 aa, Homo sapiens
  MOD_RES (17)..(17): Deiminated arginine residue
KIFVGGIKEDTEEHHLXDYFEQYGKI

SEQ ID NO: 58 - protein, 13 aa, Homo sapiens
  MOD_RES (9)..(9): Deiminated arginine residue
KNIYSEELXETKR

SEQ ID NO: 59 - protein, 21 aa, Homo sapiens
  MOD_RES (7)..(7): Deiminated arginine residue
KIDELEXQVLSRVNTLEEGKG

SEQ ID NO: 60 - protein, 18 aa, Homo sapiens
  MOD_RES (9)..(9): Deiminated arginine residue
KTLEIEACXGMNEALEKQ

SEQ ID NO: 61 - protein, 19 aa, Homo sapiens
  MOD_RES (8)..(8): Deiminated arginine residue
KALENPVXPFLAILGGAKV

SEQ ID NO: 62 - protein, 31 aa, Homo sapiens
  MOD_RES (12)..(12): Deiminated arginine residue
KIWHHTFYNELXVAPEEHPILLTEAPLNPKA

SEQ ID NO: 63 - protein, 13 aa, Homo sapiens
  MOD_RES (5)..(5): Deiminated arginine residue
KQLEXAQLENEKK

SEQ ID NO: 64 - protein, 33 aa, Homo sapiens
  MOD_RES (27)..(27): Deiminated arginine residue
KQQPTQFINPETPGYVGFANLPNQVHXKSVKKG

SEQ ID NO: 65 - protein, 25 aa, Homo sapiens
  MOD_RES (21)..(21): Deiminated arginine residue
KAAVEALQSQALHATSQQPLXKDKD

SEQ ID NO: 66 - protein, 12 aa, Homo sapiens
  MOD_RES (2)..(2): Deiminated arginine residue
KXNEFLGELQKK

SEQ ID NO: 67 - protein, 40 aa, Homo sapiens
  MOD_RES (18)..(18): Deiminated arginine residue
KGEQVSQNGLPAEQGSPXMAETVDTSEMVNGATEQRTSSK

SEQ ID NO: 68 - protein, 11 aa, Homo sapiens
  MOD_RES (8)..(8): Deiminated arginine residue
KPDIEREXLKA

SEQ ID NO: 69 - protein, 18 aa, Homo sapiens
  MOD_RES (10)..(10): Deiminated arginine residue
KVELQELNDXFANYIDKV

SEQ ID NO: 70 - protein, 21 aa, Homo sapiens
  MOD_RES (11)..(11): Deiminated arginine residue
KFADLSEAANXNNDALRQAKQ

SEQ ID NO: 71 - protein, 25 aa, Homo sapiens
  MOD_RES (17)..(17): Deiminated arginine residue
KAGFGTDEQIVDVVANXSNDQRQKI

SEQ ID NO: 72 - protein, 13 aa, Homo sapiens
  MOD_RES (7)..(7): Deiminated arginine residue
KVPLVQXGSANGL

SEQ ID NO: 73 - protein, 13 aa, Homo sapiens
  MOD_RES (4)..(4): Deiminated arginine residue
KLNXYNYIEGTKM

SEQ ID NO: 74 - protein, 32 aa, Homo sapiens
  MOD_RES (5)..(5): Deiminated arginine residue
KVPSXVTNGELLAQYMADAASELGPTTPYGKT

SEQ ID NO: 75 - protein, 15 aa, Homo sapiens
  MOD_RES (10)..(10): Deiminated arginine residue
KLLQDFFNGXDLNKS

SEQ ID NO: 76 - protein, 18 aa, Homo sapiens
  MOD_RES (6)..(6): Deiminated arginine residue
KAQALXDNSTMGYMAAKK

SEQ ID NO: 77 - protein, 22 aa, Homo sapiens
  MOD_RES (16)..(16): Deiminated arginine residue
KVEDVSAVEIVGGATXIPAVKE

SEQ ID NO: 78 - protein, 30 aa, Homo sapiens
  MOD_RES (17)..(17): Deiminated arginine residue
KAGAGSATLSMAYAGAXFVFSLVDAMNGKE

SEQ ID NO: 79 - protein, 49 aa, Homo sapiens
  MOD_RES (15)..(15): Deiminated arginine residue
  MOD_RES (24)..(24): Deiminated arginine residue
MAAAAAAGAGPEMVXGQVFDVGPXYTNLSYIGEGAYGMVCSAYDNVNKV

SEQ ID NO: 80 - protein, 17 aa, Homo sapiens
  MOD_RES (3)..(3): Deiminated arginine residue
KIXQESMPFAVVGSDKE

SEQ ID NO: 81 - protein, 13 aa, Homo sapiens
  MOD_RES (2)..(2): Deiminated arginine residue
KXKQNDVFGEAEQ

SEQ ID NO: 82 - protein, 9 aa, Homo sapiens
  MOD_RES (3)..(3): Deiminated arginine residue
KSXLFDTKH

SEQ ID NO: 83 - protein, 16 aa, Homo sapiens
  MOD_RES (6)..(6): Deiminated arginine residue
KALHQXVNIVPILAKA

SEQ ID NO: 84 - protein, 16 aa, Homo sapiens
  MOD_RES (4)..(4): Deiminated arginine residue
KTVXWCAVSEHEATKC

SEQ ID NO: 85 - protein, 12 aa, Homo sapiens
  MOD_RES (9)..(9): Deiminated arginine residue
KVVTPGASXLKA

SEQ ID NO: 86 - protein, 28 aa, Homo sapiens
  MOD_RES (18)..(18): Deiminated arginine residue
KSQSLTNAFNLPEPAPPXPSLSQDEVKA

SEQ ID NO: 87 - protein, 23 aa, Homo sapiens
  MOD_RES (4)..(4): Deiminated arginine residue
KNLXVGNGFEEGTTQGPLINEKA

SEQ ID NO: 88 - protein, 23 aa, Homo sapiens
  MOD_RES (4)..(4): Deiminated arginine residue
KNAXEITQDTNDITYADLNLPKG

SEQ ID NO: 89 - protein, 18 aa, Homo sapiens
  MOD_RES (10)..(10): Deiminated arginine residue
KEDAANNYAXGHYTIGKE

SEQ ID NO: 90 - protein, 23 aa, Homo sapiens
  MOD_RES (14)..(14): Deiminated arginine residue
KRLQTSSVLVSGLXGLGVEIAKN

SEQ ID NO: 91 - protein, 13 aa, Homo sapiens
  MOD_RES (4)..(4): Deiminated arginine residue
KYMXALDEYYDKH

SEQ ID NO: 92 - protein, 15 aa, Homo sapiens
  MOD_RES (7)..(7): Deiminated arginine residue
KTLFILXGLPGSGKS

SEQ ID NO: 93 - protein, 16 aa, Homo sapiens
  MOD_RES (4)..(4): Deiminated arginine residue
  MOD_RES (13)..(13): Deiminated arginine residue
KELXQFVPGDEPXEKM

SEQ ID NO: 94 - protein, 7 aa, Homo sapiens
  MOD_RES (5)..(5): Deiminated arginine residue
KIFFXKM

SEQ ID NO: 95 - protein, 17 aa, Homo sapiens
  MOD_RES (5)..(5): Deiminated arginine residue
KGGSXGEEVGELSRGKL

SEQ ID NO: 96 - protein, 13 aa, Homo sapiens
  MOD_RES (5)..(5): Deiminated arginine residue
KTAWXLDCAQLKE

SEQ ID NO: 97 - protein, 20 aa, Homo sapiens
  MOD_RES (6)..(6): Deiminated arginine residue
KEVFTXFCTGLTQIETLFKS

SEQ ID NO: 98 - protein, 23 aa, Homo sapiens
  MOD_RES (3)..(3): Deiminated arginine residue
KLXFPAEDEFPDLSAHNNHMAKV

SEQ ID NO: 99 - protein, 23 aa, Homo sapiens
  MOD_RES (11)..(11): Deiminated arginine residue
KDLFDPIIEDXHGGYKPSDEHKT

SEQ ID NO: 100 - protein, 41 aa, Homo sapiens
  MOD_RES (23)..(23): Deiminated arginine residue
KRGTGGVDTAAVGGVFDVSNADXLGFSEVELVQMVVDGVKL

SEQ ID NO: 101 - protein, 25 aa, Homo sapiens
  MOD_RES (17)..(17): Deiminated arginine residue
KDNFTLIPEGTNGTEEXMSVIWDKA

SEQ ID NO: 102 - protein, 15 aa, Homo sapiens
  MOD_RES (5)..(5): Deiminated arginine residue
  MOD_RES (10)..(10): Deiminated arginine residue
KNIPXITSDXLLIKG

SEQ ID NO: 103 - protein, 30 aa, Homo sapiens
  MOD_RES (18)..(18): Deiminated arginine residue
KTHNSSLEYNIFEGMECXGSPLVVISQGKI

SEQ ID NO: 104 - protein, 16 aa, Homo sapiens
  MOD_RES (9)..(9): Deiminated arginine residue
KELSDIALXIVAPGKG

SEQ ID NO: 105 - protein, 16 aa, Homo sapiens
  MOD_RES (10)..(10): Deiminated arginine residue
KDDNGVPFVXTIQDKG

SEQ ID NO: 106 - protein, 30 aa, Homo sapiens
  MOD_RES (24)..(24): Deiminated arginine residue
KGVVPLAGTDGETTTQGLDGLSEXCAQYKK

SEQ ID NO: 107 - protein, 15 aa, Homo sapiens
  MOD_RES (10)..(10): Deiminated arginine residue
KDGADFAKWXCVLKI

SEQ ID NO: 108 - protein, 49 aa, Homo sapiens
  MOD_RES (5)..(5): Deiminated arginine residue
  MOD_RES (21)..(21): Deiminated arginine residue
KISEXTPSALAILENANVLAXYASICQQNGIVPIVEPEILPDGDHDLKR

SEQ ID NO: 109 - protein, 14 aa, Homo sapiens
  MOD_RES (2)..(2): Deiminated arginine residue
KXAEVNGLAAQGKY

SEQ ID NO: 110 - protein, 44 aa, Homo sapiens
  MOD_RES (13)..(13): Deiminated arginine residue
KAGAAERELPLYXHIAQLAGNSDLILPVPAFNVINGGSHAGNKL

SEQ ID NO: 111 - protein, 26 aa, Homo sapiens
  MOD_RES (4)..(4): Deiminated arginine residue
KEIXTQYEAMASSNMHEAEEWYRSKF

SEQ ID NO: 112 - protein, 19 aa, Homo sapiens
  MOD_RES (6)..(6): Deiminated arginine residue
KDEMAXHLQEYQDLLNVKL

SEQ ID NO: 113 - protein, 44 aa, Homo sapiens; identical to SEQ ID NO: 110
  MOD_RES (13)..(13): Deiminated arginine residue

SEQ ID NO: 114 - protein, 13 aa, Homo sapiens
  MOD_RES (5)..(5): Deiminated arginine residue
KLIPXNTVVPTKK

SEQ ID NO: 115 - protein, 26 aa, Homo sapiens
  MOD_RES (22)..(22): Deiminated arginine residue
KDLYANTVLSGGTTMYPGIADXMQKE

SEQ ID NO: 116 - protein, 16 aa, Homo sapiens
  MOD_RES (7)..(7): Deiminated arginine residue
KYNQLLXIEEELGSKA

SEQ ID NO: 117 - protein, 16 aa, Homo sapiens
  MOD_RES (4)..(4): Deiminated arginine residue
KDCXYALYDATYETKE

SEQ ID NO: 118 - protein, 19 aa, Homo sapiens
  MOD_RES (3)..(3): Deiminated arginine residue
KEXITEQPLKEEEDEDRKN

SEQ ID NO: 119 - protein, 13 aa, Homo sapiens
  MOD_RES (9)..(9): Deiminated arginine residue
KEALLQASXDQKK

SEQ ID NO: 120 - protein, 20 aa, Homo sapiens
  MOD_RES (7)..(7): Deiminated arginine residue
KIPKQYXTKETTFGATLSKD

SEQ ID NO: 121 - protein, 20 aa, Homo sapiens
  MOD_RES (12)..(12): Deiminated arginine residue
KEGMNIVEAMEXFGSRNGKT

SEQ ID NO: 122 - protein, 30 aa, Homo sapiens
  MOD_RES (16)..(16): Deiminated arginine residue
KMNQLLSRTPALSPQXPLTTQQPQSGTLKD

SEQ ID NO: 123 - protein, 24 aa, Homo sapiens
  MOD_RES (3)..(3): Deiminated arginine residue
KYXAAHVFFTDSCPDALFNELVKS

SEQ ID NO: 124 - protein, 20 aa, Homo sapiens
  MOD_RES (4)..(4): Deiminated arginine residue
KLCXVEQDLAMGTDAEGEKI

SEQ ID NO: 125 - protein, 21 aa, Homo sapiens
  MOD_RES (8)..(8): Deiminated arginine residue
KDMAAVQXTLMALGSLAVTKN

SEQ ID NO: 126 - protein, 18 aa, Homo sapiens
  MOD_RES (10)..(10): Deiminated arginine residue
KEDAANNYAXGHYTIGKE

SEQ ID NO: 127 - protein, 26 aa, Homo sapiens
  MOD_RES (4)..(4): Deiminated arginine residue
KVQXAVCMLSNTTAIAEAWARLDHKF

SEQ ID NO: 128 - protein, 21 aa, Homo sapiens
  MOD_RES (8)..(8): Deiminated arginine residue
KTGGAVDXLTDTSRYTGSHKE

SEQ ID NO: 129 - protein, 19 aa, Homo sapiens
  MOD_RES (6)..(6): Deiminated arginine residue
KNGQTXEHALLAYTLGVKQ

SEQ ID NO: 130 - protein, 37 aa, Homo sapiens
  MOD_RES (12)..(12): Deiminated arginine residue
KIGGIGTVPVGXVETGILRPGMVVTFAPVNITTEVKS

SEQ ID NO: 131 - protein, 12 aa, Homo sapiens
  MOD_RES (6)..(6): Deiminated arginine residue
KSTLAXVIVDKY

SEQ ID NO: 132 - protein, 16 aa, Homo sapiens
  MOD_RES (7)..(7): Deiminated arginine residue
KITPGAXGAFSEEYKR

SEQ ID NO: 133 - protein, 11 aa, Homo sapiens
  MOD_RES (3)..(3): Deiminated arginine residue
KVXFLEQQNKA

SEQ ID NO: 134 - protein, 14 aa, Homo sapiens
  MOD_RES (11)..(11): Deiminated arginine residue
KALAAELNQLXAKE

SEQ ID NO: 135 - protein, 21 aa, Homo sapiens
  MOD_RES (17)..(17): Deiminated arginine residue
KFADLTDAAARNAELLXQAKH

SEQ ID NO: 136 - protein, 13 aa, Homo sapiens
  MOD_RES (6)..(6): Deiminated arginine residue
KTVEMXDGEVIKE

SEQ ID NO: 137 - protein, 19 aa, Homo sapiens
  MOD_RES (8)..(8): Deiminated arginine residue
KDSHHPAXTAHYGSLPQKS

SEQ ID NO: 138 - protein, 18 aa, Homo sapiens
  MOD_RES (5)..(5): Deiminated arginine residue
KSHGXTQDENPVVHFFKN

SEQ ID NO: 139 - protein, 16 aa, Homo sapiens
  MOD_RES (7)..(7): Deiminated arginine residue
KNIVTPXTPPPSQGKG

SEQ ID NO: 140 - protein, 17 aa, Homo sapiens
  MOD_RES (10)..(10): Deiminated arginine residue
KAISSPTVSXLTDTTKF

SEQ ID NO: 141 - protein, 21 aa, Homo sapiens
  MOD_RES (6)..(6): Deiminated arginine residue
KGIAGXQDILDDSGYVSAYKN

SEQ ID NO: 142 - protein, 13 aa, Homo sapiens
  MOD_RES (4)..(4): Deiminated arginine residue
KPLXLPLQDVYKI

SEQ ID NO: 143 - protein, 28 aa, Sus sp.
  MOD_RES (14)..(14): Deiminated arginine residue
LVQQPQNVSTFINXPALGILPPENFVEK

SEQ ID NO: 144 - protein, 9 aa, Sus sp.
  MOD_RES (3)..(3): Deiminated arginine residue
LNXPTLSEK

SEQ ID NO: 145 - protein, 21 aa, Sus sp.
  MOD_RES (14)..(14): Deiminated arginine residue
ITIPVQTFSNLQIXETSLDTK

SEQ ID NO: 146 - protein, 11 aa, Sus sp.
  MOD_RES (5)..(5): Deiminated arginine residue
TVEMXDGEVIK

SEQ ID NO: 147 - protein, 18 aa, Sus sp.
  MOD_RES (11)..(11): Deiminated arginine residue
AFLASPEHVNXPINGNGK

SEQ ID NO: 148 - protein, 31 aa, Sus sp.
  MOD_RES (9)..(9): Deiminated arginine residue
SGLIYEETXGVLKVFLENVIRDAVTYTEHAK

SEQ ID NO: 149 - protein, 21 aa, Sus sp.
  MOD_RES (5)..(5): Deiminated arginine residue
TVETXDGQVINETSQHHDDLE

SEQ ID NO: 150 - protein, 522 aa, Artificial Sequence
  Description of Artificial Sequence Synthetic polypeptide
  MOD_RES (153)..(153): Deiminated arginine residue
  MOD_RES (268)..(268): Deiminated arginine residue
MALLPRALSAGAGPSWRRAARAFRGFLLLLPEPAALTRALSRAMACRQEPQPQGPPPAAGAVASYDYLVIGGGSGGLASARRAAELGARAAVVESHKLGGTCVNVGCVPKKVMWNTAVHSEFMHDHADYGFPSCEGKFNWRVIKEKRDAYVSXLNAIYQNNLTKSHIEIIRGHAAFTSDPKPTIEVSGKKYTAPHILIATGGMPSTPHESQIPGASLGITSDGFFQLEELPGRSVIVGAGYIAVEMAGILSALGSKTSLMIRHDKVLXSFDSMISTNCTEELENAGVEVLKFSQVKEVKKTLSGLEVSMVTAVPGRLPVMTMIPDVDCLLWAIGRVPNTKDLSLNKLGIQTDDKGHIIVDEFQNTNVKGIYAVGDVCGKALLTPVAIAAGRKLAHRLFEYKEDSKLDYNNIPTVVFSHPPIGTVGLTEDEAIHKYGIENVKTYSTSFTPMYHAVTKRKTKCVMKMVCANKEEKVVGIHMQGLGCDEMLQGFAVAVKMGATKADFDNTVAIHPTSSEELVTLR

SEQ ID NO: 151 - protein, 9 aa, Unknown
  Description of Unknown Core sequence
  MOD_RES (3)..(3): Deiminated arginine residue
EMXDGEVIK

SEQ ID NO: 152 - protein, 10 aa, Sus sp.
  MOD_RES (3)..(3): Deiminated arginine residue
LNXPLTLSEK